SEQUENCE LISTING (33 sequences)

SEQ ID NO: 1 - protein, 41 aa, synthetic construct
  REGION 1..41: note = Synthetic
TEPQEESEEEVEEPEERQQTPEVVPDDSGTFYDQTVSNDLE

SEQ ID NO: 2 - protein, 41 aa, synthetic construct
  REGION 1..41: note = Synthetic
TDPQDDSDDDVDDPDDRQQTPDVVPDDSGTFYDQTVSNDLD

SEQ ID NO: 3 - protein, 41 aa, synthetic construct
  REGION 1..41: note = Synthetic
TKPQKKSKKKVKKPKKRQQTPKVVPDDSGTFYDQTVSNDLK

SEQ ID NO: 4 - protein, 41 aa, synthetic construct
  REGION 1..41: note = Synthetic
TRPQRRSRRRVRRPRRRQQTPRVVPDDSGTFYDQTVSNDLR

SEQ ID NO: 5 - protein, 41 aa, synthetic construct
  REGION 1..41: note = Synthetic
TQPQQQSQQQVQQPQQRQQTPQVVPDDSGTFYDQTVSNDLQ

SEQ ID NO: 6 - protein, 32 aa, synthetic construct
  REGION 1..32: note = Synthetic
TEPQEESEEEVEEPEERQQTPEVVPDDSGTFY

SEQ ID NO: 7 - protein, 27 aa, synthetic construct
  REGION 1..27: note = Synthetic
TEPQEESEEEVEEPEERQQTPEVVPDD

SEQ ID NO: 8 - protein, 43 aa, synthetic construct
  REGION 1..43: note = Synthetic
ELDEESEDEVEEEQEDRQPSPEPVQENANSAYYDAHPVTNGIE

SEQ ID NO: 9 - protein, 55 aa, synthetic construct
  REGION 1..55: note = Synthetic
KEEVDEDRDVDESSPQDSPPSKASPAQDGRPPQTAAREATSIPGFPAEGAIPLPV

SEQ ID NO: 10 - protein, 21 aa, synthetic construct
  REGION 1..21: note = Synthetic
EGEVAEEPNSRPQEKSEQDLE

SEQ ID NO: 11 - protein, 17 aa, synthetic construct
  REGION 1..17: note = Synthetic
  VARIANT 2: note = X is D, E, K or R
  VARIANT 5: note = X is D, E, K or R
  VARIANT 6: note = X is D, E, K or R
  VARIANT 8: note = X is D, E, K or R
  VARIANT 9: note = X is D, E, K or R
  VARIANT 10: note = X is D, E, K or R
  VARIANT 12: note = X is D, E, K or R
  VARIANT 13: note = X is D, E, K or R
  VARIANT 15: note = X is D, E, K or R
  VARIANT 16: note = X is D, E, K or R
  VARIANT 17: note = X is D, E, K or R
TXPQXXSXXXVXXPXXR

SEQ ID NO: 12 - protein, 17 aa, synthetic construct
  REGION 1..17: note = Synthetic
  VARIANT 1: note = X is D, E, K or R
  VARIANT 3: note = X is D, E, K or R
  VARIANT 4: note = X is D, E, K or R
  VARIANT 5: note = X is D, E, K or R
  VARIANT 7: note = X is D, E, K or R
  VARIANT 8: note = X is D, E, K or R
  VARIANT 9: note = X is D, E, K or R
  VARIANT 11: note = X is D, E, K or R
  VARIANT 12: note = X is D, E, K or R
  VARIANT 13: note = X is D, E, K or R
  VARIANT 15: note = X is D, E, K or R
  VARIANT 16: note = X is D, E, K or R
  VARIANT 17: note = X is D, E, K or R
XLXXXSXXXVXXXQXXX

SEQ ID NO: 13 - skipped

SEQ ID NO: 14 - protein, 9 aa, synthetic construct
  REGION 1..9: note = Synthetic
  VARIANT 1: note = X is D, E, K or R
  VARIANT 2: note = X is D, E, K or R
  VARIANT 4: note = X is D, E, K or R
  VARIANT 7: note = X is D, E, K or R
  VARIANT 9: note = X is D, E, K or R
XXSXVQXLX

SEQ ID NO: 15 - protein, 17 aa, synthetic construct
  REGION 1..17: note = Synthetic
TEPQEESEEEVEEPEER

SEQ ID NO: 16 - protein, 17 aa, synthetic construct
  REGION 1..17: note = Synthetic
TDPQDDSDDDVDDPDDR

SEQ ID NO: 17 - protein, 17 aa, synthetic construct
  REGION 1..17: note = Synthetic
TKPQKKSKKKVKKPKKR

SEQ ID NO: 18 - protein, 17 aa, synthetic construct
  REGION 1..17: note = Synthetic
TRPQRRSRRRVRRPRRR

SEQ ID NO: 19 - protein, 17 aa, synthetic construct
  REGION 1..17: note = Synthetic
ELDEESEDEVEEEQEDR

SEQ ID NO: 20 - protein, 12 aa, synthetic construct
  REGION 1..12: note = Synthetic
KEEVDEDRDVDE

SEQ ID NO: 21 - protein, 8 aa, synthetic construct
  REGION 1..8: note = Synthetic
EKSEQDLE

SEQ ID NO: 22 - protein, 11 aa, synthetic construct
  REGION 1..11: note = Synthetic
TFYDQTVSNDL

SEQ ID NO: 23 - protein, 15 aa, synthetic construct
  REGION 1..15: note = Synthetic
ANSAYYDAHPVTNGI

SEQ ID NO: 24 - protein, 25 aa, synthetic construct
  REGION 1..25: note = Synthetic
PPQTAAREATSIPGFPAEGAIPLPV

SEQ ID NO: 25 - protein, 12 aa, synthetic construct
  REGION 1..12: note = Synthetic
EGEVAEEPNSRP

SEQ ID NO: 26 - protein, 11 aa, synthetic construct
  REGION 1..11: note = Synthetic
GGRKKRRQRRR

SEQ ID NO: 27 - protein, 17 aa, synthetic construct
  REGION 1..17: note = Synthetic
RQIKIWFQNRRMKWKKK

SEQ ID NO: 28 - protein, 6 aa, synthetic construct
  REGION 1..6: note = Synthetic
SGRPVL

SEQ ID NO: 29 - protein, 14 aa, synthetic construct
  REGION 1..14: note = Synthetic
GAPGSAGSAAGGSG

SEQ ID NO: 30 - protein, 7 aa, synthetic construct
  REGION 1..7: note = Synthetic
ENLVFQG

SEQ ID NO: 31 - protein, 41 aa, synthetic construct
  REGION 1..41: note = Synthetic
  VARIANT 2: note = X is D, E, or K
  VARIANT 5: note = X is D, E, or K
  VARIANT 22: note = X is D, E, or K
  VARIANT 41: note = X is D, E, or K
  VARIANT 6: note = X is D, E, or K
  VARIANT 8: note = X is D, E, or K
  VARIANT 9: note = X is D, E, or K
  VARIANT 10: note = X is D, E, or K
  VARIANT 12: note = X is D, E, or K
  VARIANT 13: note = X is D, E, or K
  VARIANT 15: note = X is D, E, or K
  VARIANT 16: note = X is D, E, or K
TXPQXXSXXXVXXPXXRQQTPXVVPDDSGTFYDQTVSNDLX

SEQ ID NO: 32 - protein, 60 aa, synthetic construct
  REGION 1..60: note = Synthetic
TEPQEESEEEVEEPEERQQTPEVVPDDSGTFYDQTVSNDLEGGRKKRRQRRRHHHHHHHH

SEQ ID NO: 33 - protein, 70 aa, synthetic construct
  REGION 1..70: note = Synthetic
RQIKIWFQNRRMKWKKKTKPQKKSKKKVKKPKKRQQTPKVVPDDSGTFYDQTVSNDLKSGRPVLHHHHHH

(19) United States
(12) Patent Application Publication
(10) Pub. No.: US 2025/0352611 A1
(43) Pub. Date: Nov. 20, 2025

(54) METHODS FOR TREATING CATARACTS USING POLYPEPTIDES

(71) Applicant: REJUKON BIOPHARM INC., Shanghai (CN)

(72) Inventor: Chenchen WANG, Shanghai (CN)

(21) Appl. No.: 18/836,758

(22) PCT Filed: Feb. 8, 2023
(86) PCT No.: PCT/CN2023/075042

§ 371 Date: Aug. 8, 2024

(30) Foreign Application Priority Data
Feb. 9, 2022 (WO) PCT/CN2022/075738

(51) Int. Cl. A61K 38/16

(52) U.S. Cl. CPC A61K 38/16

(57) ABSTRACT

Provided is a polypeptide capable of dissolving protein aggregates. Also provided is a method of treating a phase separation associated diseases, such as phase separation associated visual disorders (e.g., cataracts) using the polypeptide provided herein.

### CROSS-REFERENCE TO RELATED APPLICATIONS

This application is a national stage application under 35 U.S.C. § 371 of PCT International Application No. PCT/CN2023/075042, filed on Feb. 8, 2023, which claims the benefits of PCT International Application No. PCT/CN2022/075738 filed on Feb. 9, 2022, which is hereby incorporated by reference in their entireties.

### SEQUENCE LISTING

The sequence listing that is contained in the file named “081734-8003US01_seql.xml”, which is 56,584 bytes and was created on Mar. 27, 2025, is filed herewith by electronic submission and is incorporated by reference herein.

### FIELD OF THE INVENTION

The present invention generally relates to phase separation-associated diseases, such as cataracts. In particular, the present invention relates to compositions and methods for dissolving and/or preventing the formation of crystallin protein aggregates, such as βD-crystallin aggregate, γD-crystallin aggregate, or a combination thereof.

### BACKGROUND OF THE INVENTION

Cataract is the number one cause of blindness and severe visual impairment worldwide. The lens is an important part of the eye's refractive system. Cataracts occur if part or all of the lens is clouded due to various reasons. The internationally recognized rapid and effective treatment for cataracts is surgery, in which the patient's cloudy lens is removed and an intraocular lens is implanted. However, in general, the cost of surgical treatment is relatively high, which is a great economic burden for patients. With the prolongation of human life expectancy and the emergence of an aging population, this problem is more prominent.

Therefore, needs exist for effective, safe and cheap treatment for cataract.

### BRIEF SUMMARY OF THE INVENTION

Provided herein are methods for treating phase separation associated disease (e.g., phase separation associated visual disorders, such as cataracts) using polypeptides capable of reversing phase separation.

In one aspect, provided herein is a method for treating phase separation associated disease in a subject in need thereof, comprising administering to the subject in need thereof a therapeutically effective amount of a polypeptide, a polynucleotide encoding the polypeptide, and/or a vector comprising the polynucleotide, the polypeptide comprising a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues, and wherein the polypeptide is capable of reversing phase separation.

In certain embodiments, the phase separation associated disease is phase separation associated vision disorder.

In certain embodiments, the phase separation associated vision disorder is cataract.

In certain embodiments, the hydrophilic segment has a sequence selected from the group consisting of:

(SEQ ID NO: 11)
TX₁PQX₁X₁SX₁X₁X₁VX₁X₁PX₁X₁R,
(SEQ ID NO: 12)
X₁LX₁X₁X₁SX₁X₁X₁VX₁X₁X₁QX₁X₁X₁,
(SEQ ID NO: 13)
X₁X₁X₁VX₁X₁X₁X₁X₁VX₁X₁,
and
(SEQ ID NO: 14)
X₁X₁SX₁VQX₁LX₁,

wherein each X₁ is respectively Asp, Glu, Lys or Arg.

In certain embodiments, the hydrophilic segment has a sequence selected from the group consisting of:

(SEQ ID NO: 15)
TEPQEESEEEVEEPEER,
(SEQ ID NO: 16)
TDPQDDSDDDVDDPDDR,
(SEQ ID NO: 17)
TKPQKKSKKKVKKPKKR,
(SEQ ID NO: 18)
TRPQRRSRRRVRRPRRR,
(SEQ ID NO: 19)
ELDEESEDEVEEEQEDR,
(SEQ ID NO: 20)
KEEVDEDRDVDE,
and
(SEQ ID NO: 21)
EKSEQDLE,

or a sequence having at least 90% identity thereto, or a sequence having 1, 2, 3, 4, or 5 amino acid residue difference therefrom.

In certain embodiments, the hydrophobic segment has a sequence selected from the group consisting of:

(SEQ ID NO: 22)
TFYDQTVSNDL,
(SEQ ID NO: 23)
ANSAYYDAHPVTNGI,
(SEQ ID NO: 24)
PPQTAAREATSIPGFPAEGAIPLPV,
and
(SEQ ID NO: 25)
EGEVAEEPNSRP,

or a sequence having at least 90% identity thereto, or a sequence having 1, 2, 3, 4, or 5 amino acid residue difference therefrom.

In certain embodiments, the polypeptide comprises a sequence selected from the group consisting of:

(SEQ ID NO: 1)
TEPQEESEEEVEEPEERQQTPEVVPDDSGTFYDQTVSNDLE
(RJK001),
(SEQ ID NO: 2)
TDPQDDSDDDVDDPDDRQQTPDVVPDDSGTFYDQTVSNDLD
(RJK002),
(SEQ ID NO: 3)
TKPQKKSKKKVKKPKKRQQTPKVVPDDSGTFYDQTVSNDLK
(RJK012),
(SEQ ID NO: 4)
TRPQRRSRRRVRRPRRRQQTPRVVPDDSGTFYDQTVSNDLR,
(SEQ ID NO: 8)
ELDEESEDEVEEEQEDRQPSPEPVQENANSAYYDAHPVINGIE,
(SEQ ID NO: 9)
KEEVDEDRDVDESSPQDSPPSKASPAQDGRPPQTAAREATSIPGFPAEG
AIPLPV,
and
(SEQ ID NO: 10)
EGEVAEEPNSRPQEKSEQDLE,

or a sequence having at least 90% identity thereto, or a sequence having 1, 2, 3, 4, or 5 amino acid residue difference therefrom.

In certain embodiments, the hydrophilic segment has a length of 10-17 amino acid residues among which at least 60% are Asp, Glu, Lys, or Arg,
wherein the hydrophobic segment having a length of 10-12 amino acid residues among which at least 35% are Tyr, Phe, Leu, or Val, and
wherein the polypeptide has a length of 20-28 amino acid residues.

In certain embodiments, the hydrophilic segment has a sequence of:

(SEQ ID NO: 11)
TX₁PQX₁X₁SX₁X₁X₁VX₁X₁PX₁X₁R,

wherein each X₁ is Asp, Glu or Lys.

In certain embodiments, the hydrophobic segment has a sequence of:

(SEQ ID NO: 22)
TFYDQTVSNDL,

or a sequence having at least 90% identity thereto, or a sequence having 1, 2, 3, 4, or 5 amino acid residue difference therefrom.

In certain embodiments, the polypeptide comprises a sequence of:

(SEQ ID NO: 31)
TX₁PQX₁X₁SX₁X₁X₁VX₁X₁PX₁X₁RQQTPX₁VVPDDSGTFYDQTVSNDLX₁,

wherein each X₁ is Asp, Glu or Lys.

In certain embodiments, the polypeptide is fused with a cell-penetrating peptide.

In certain embodiments, the cell-penetrating peptide comprises a sequence selected from the group consisting of:

(SEQ ID NO: 26)
GGRKKRRQRRR,
(SEQ ID NO: 27)
RQIKIWFQNRRMKWKKK

In certain embodiments, the cell-penetrating peptide is fused at the N-terminus or C-terminus of the polypeptide.

In certain embodiments, the polypeptide is further fused with a linker.

In certain embodiments, the linker comprises a sequence selected from the group consisting of:

(SEQ ID NO: 28)
SGRPVL,
(SEQ ID NO: 29)
GAPGSAGSAAGGSG,
and
(SEQ ID NO: 30)
ENLVFQG.

In certain embodiments, the polypeptide is further fused with a his tag.

In certain embodiments, the polynucleotide is a DNA or an RNA.

In certain embodiments, the vector is a virus vector.

In certain embodiments, the polypeptide, the polynucleotide encoding the polypeptide, and/or a vector comprising the polynucleotide is administered orally, intravenously, intramuscularly, enterally, mtraocularly, subretinally, intravitreally, topically, ocularly (eye drops, insert, injection or implant), sublingually, rectally or by injection, nasal spray or inhalation.

In certain embodiments, the polypeptide, the polynucleotide encoding the polypeptide, and/or a vector comprising the polynucleotide is administered ocularly (eye drops, insert, injection or implant).

In certain embodiments, the polypeptide, the polynucleotide encoding the polypeptide, and/or a vector comprising the polynucleotide is formulated as an ophthalmic solution, an ophthalmic ointment, an ophthalmic wash, an intraocular infusion solution, a wash for anterior chamber, an internal medicine, an injection, an intravitreal injection, an anterior chamber injection, a subarachnoid injection, or preservative for extracted cornea.

In another aspect, also provided herein is a method for dissolving a crystalline protein aggregate and/or preventing the formation of crystalline protein aggregate in a cell, comprising introducing to the cell the polypeptide comprises a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues, and wherein the polypeptide is capable of inhibiting phase separation.

In certain embodiments, the crystalline protein aggregate is βD-crystallin aggregate, γD-crystallin aggregate, or a combination thereof.

In another aspect, also provided herein is a kit for treating and phase separation associated diseases (e.g., cataracts), comprising a formulation of a therapeutically effective amount of a polypeptide, a polynucleotide encoding the polypeptide, and/or a vector comprising the polynucleotide, a pharmaceutically acceptable carrier and instructions for administering the formulation such that the administration treats the phase separation associated diseases,
wherein the polypeptide comprises a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues, and wherein the polypeptide is capable of inhibiting phase separation.

In another aspect, also provided herein is a method for inhibiting or reversing phase separation of crystallin protein, comprising contacting the crystallin protein with the polypeptide comprises a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues.

In certain embodiments, the crystalline protein aggregate is βD-crystallin aggregate, γD-crystallin aggregate, or a combination thereof.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the effect of polypeptides Champ-E, Champ-D, Champ-K and Champ-Q on dissolving G3BP1-GFP protein aggregates in vitro.

FIG. 2 shows Phase separation of crystallins is a reversible state. Left, right, at room temperature (25° C.); Middle, at 4° C.

FIG. 3 shows purified human full-length γD-crystallin verified by Coomassie Bright Blue staining.

FIG. 4 shows that Champ-E (RJK001) peptide inhibits phase separation of γD-crystallin proteins.

FIG. 5 shows ¹H-¹⁵N HSQC spectra of γD-crystallin. The two-dimensional [¹H-¹⁵N] heteronuclear single quantum coherence (HSQC) spectra were recorded to determine the domain interactions at the residue-specific level.

FIG. 6 shows overlay of the 2D ¹H-¹⁵N HSQC spectra of 300 M ¹⁵N γD-crystallin alone (dark grey) and in the presence of RJK001 at molar ratios (γD-crystallin: RJK001) of 1:2 (light grey). HSQC spectrums were acquired and analysis with ccpNMR.

FIG. 7 shows residue-specific chemical shift deviations (CSD) between γD-crystallin in the presence of RJK001 at molar ratios (γD-crystallin: RJK001) of 1:2, indicating that significant chemical shift differences are distributed across the chain after titrated with RJK001.

FIG. 8 shows the schematic diagram of the interaction of γD-crystallin protein with RJK001 (data reconstruction). The amino acids with chemical shift differences higher than 0.1 was highlighted in the structure (2KFB), arrows indicate the interaction of crystallin and RJK001.

FIG. 9 shows quantitative effect of RJK001 and RJK012 peptides fused with cell penetrating peptides on disaggregation of G3BP1 proteins in vitro.

FIG. 10 shows ratio and half-life of Champ peptides fused with cell-penetrating peptide transferred into eukaryotic cells.

FIG. 11 shows live imaging and analysis of Champ peptides (e.g., RJK001 and RJK012) affect the aggregation of γD (W43R) in eukaryotic cells.

FIG. 12 shows the photographs of a cataractous human lens lysates treated with RJK001 peptides showing increased lens clarity.

FIG. 13 shows the effect of Champ peptides on the re-dissolution of crystallin protein aggregates from different degrees of human cataract patients (e.g., C1N2P0) by ThT fluorescence in vitro. DM: diabetes mellitus. lens opacity classification: C: cortex; N: nucleus; P: the posterior lens capsule.

FIG. 14 shows increase in soluble crystallin protein from human cataract patients by co-incubation of Champ-E(RJK001) peptides but not protein buffer. Quantitative analysis was performed using densitometry of crystallin proteins by western blot analysis of the supernatant or insoluble fraction of cell lysates.

FIG. 15 shows that the photographs of a cataractous human lens treated with C-terminal fusion cell-penetrating peptide RJK001 peptides showing increased lens clarity.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### I. Definition

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

As used herein, the term “G3BP1” refers to a tunable switch that triggers phase separation to assemble stress granules, for example, that triggers RNA-dependent liquid-liquid phase separation (LLPS) in response to a rise in intracellular free RNA concentrations.

As used herein, the term “administering” means providing a pharmaceutical agent or composition to a subject, and includes, but is not limited to, administering by a medical professional and self-administering.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure.

As used herein, the term “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself. In certain embodiments, the therapeutically effective amount of the polypeptide provided herein in an ophthalmic pharmaceutical formulation provided herein is at relatively low concentrations in liquid drops, e.g., at least 10⁻⁹ M, at least 0.5 to 1×10⁻⁸ M, at least 0.5 to 1×10⁻⁷ M, at least 0.5 to 1×10⁻⁶ M, at least 0.5 to 1×10⁻⁵ M, at least 0.5 to 1×10⁻⁴ M, at least 0.5 to 1×10³ M, at least 0.5 to 1×10⁻² M, at least 0.5 to 1×10⁻¹ M, or at least 0.5 to 1 M or any concentration falling in a range between these values (e.g., 10⁻⁹ M to 1 M), may reverse such vision disorders with only one, two, three or multiple, daily applications and does so rapidly.

The term “host cell” means a cell that has been transformed, or is capable of being transformed, with a nucleic acid sequence and thereby expresses a protein of interest. The term includes the progeny of the parent cell, whether or not the progeny is identical in morphology or in genetic make-up to the original parent cell, so long as the gene of interest is present.

The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool or may customize the parameters as appropriate for the alignment, such as for example, by selecting a suitable algorithm.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence) or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally occurring amino acid and polymers.

The phrase “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “protein aggregate” used herein refers the aggregation of a protein which appears either intra or extracellularly. In some embodiments, the protein is an intrinsically disordered protein or a mis-folded protein. In some embodiments, the aggregation occurs when the concentration of the protein exceeds the solubility of the protein. In some embodiments, the concentration of the protein exceeds the thermodynamic solubility, but the protein remains in a metastable liquid-like state by buffering via heterotypic interactions. Disturbance of metastable form of the protein would cause the loss of protein solubility and lead to protein aggregation.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder. For example, the subject may have or is at risk for developing a cataract. Subjects at risk of developing a cataract include, without limitation, subjects with a mutation linked to a cataract, subjects with a family history of developing cataracts, subjects exposed to radiation, diabetics, and the like.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. It should be understood that “treating” a vision disorder does not require a 100% abolition or reversal of a vision disorder. In certain embodiments, “treating” vision disorders by the methods provided herein alleviates, inhibits, prevents and/or reverses dysfunction of the lens, e.g., lens nucleus opacity (N), cortical opacity (C), posterior subcapsular opacity (P) and lens nucleus color (NC) by, e.g., at least about 5%, at least about 10% or at least about 20% compared to levels observed in the absence of the ophthalmic pharmaceutical composition or method provided herein (e.g., in a biologically matched control subject or specimen that is not exposed to the ophthalmic pharmaceutical composition or method provided herein.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. Methods of Treating Phase Separation Associated Disease

Phase separation, also known as phase transition, is a concept originally derived from physical chemistry. Phase refers to the part of a system with completely uniform physical and chemical properties. When the two phases are mixed, such as oil dripping into water, the phenomenon of two-phase separation will occur. For cells with complex components, certain proteins and nucleic acid molecules can be combined through multivalent interactions, thereby spontaneously forming another phase with different physical and chemical properties from the original environment—this phenomenon is called “intracellular and biological phase separation”.

The concept of “phase separation” was introduced into biological structure assembly for the first time in the explanation of the liquid-like behavior of P particles in the study of P granules in C. elegans by Brangwynne and Hyman 2009 (see, Brangwynne, Clifford P., et al. “Germline P granules are liquid droplets that localize by controlled dissolution condensation.” Science 324.5935 (2009): 1729-1732). Research in recent years has shown that phase separation plays an important role in the assembly of membraneless organelles, signaling complexes, cytoskeletons, etc., and phase separation has been observed in membraneless organelles, including stress granules, nucleoli formation of the structure. Changes in physiologically relevant conditions can also alter the homeostasis formed by phase separation. In cells, phase separation can be caused by, for example, starvation-induced pH reduction, viscosity change, or increase in calcium ion and other high-valent cation concentrations, changes in the expression level of the protein itself, and phosphorylation modifications. For non-isothermal organisms, such as yeast and nematodes, changes in temperature may also trigger intracellular protein phase separation. In other words, phase separation is ubiquitous in cells.

Abnormal phase separation of specific proteins may form structures that are more stable and difficult to reverse and may be the cause of specific diseases. There is substantial evidence for the link between membrane-free structures formed by phase separation and disease. As early as the 1970s, scientists proposed that cataracts are a phase-separation disease, the molecular mechanism of which is the protein aggregation.

Most of the proteins that form the human lens are crystallins, and whether the lens can work properly depends on these crystallins. The most abundant proteins among crystallins are CRYAA and CRYAB, which are produced under stress or damage and function as “chaperones”, i.e., recognizing damaged and misfolded proteins in the lens and interacting with them to prevent them from clumping together. But with age, there are more and more misfolded proteins, and these molecular chaperones will have problems themselves and accumulate, so the proteins will accumulate more and more, and eventually cataracts will form.

Cataract is a degenerative disease in which the optical quality of the lens decreases due to reduced transparency or color changes. Aging, heredity, local nutritional disorders, immune and metabolic abnormalities, as well as trauma, poisoning, radiation, etc. can cause lens metabolic disorders, as well as a variety of reasons that leads to lens opacity can cause cataracts. In order to maintain vision, the lens must remain transparent to visible light. The lens is avascular, with no arterial or venous circulation. During the maturation of fibroblasts, organelles, including the nucleus, mitochondria, endoplasmic reticulum, ribosomes, and other organelles, gradually disappear, thereby reducing light scattering. Lensin expression is highly upregulated during differentiation, reaching 90% in mature lenses. At concentrations of 250-400 mg/ml, the short-range ordered packing of crystallins helps improve the clarity of concentrated solutions, while the polydisperse mixture of crystallins avoids crystallization. Since mature fibroblasts within the lens nucleus lack the protein synthesis and degradation machinery necessary to remove and replace damaged proteins, the main requirements for lens proteins are excellent solubility and long-term stability in their native conformation.

Lens proteins accumulate polypeptide chain instability over time due to the accumulation of covalent modifications such as proteolytic activity, non-enzymatic modifications, and oxidative damage. Proteomic analysis of crystallins has identified several damage-related covalent modifications, including deamidation, oxidation, glycosylation, and truncation. Instability due to long-term accumulation of covalent modifications/damages may promote partial protein unfolding, leading to the formation of intermediate conformations that expose previously buried hydrophobic residues and allow crystallins to phase separate. The early stages of cataract development are associated with phase separation of the lens cytoplasm.

Phase separation is reversible and is characterized by the phase separation critical temperature, Tc, at which the cytoplasm undergoes a transition from a transparent to an opaque state. In the clear state, short-range arrangements in the organization of cytoplasmic proteins allow the cytoplasm to exist as a single homogeneous phase. After intracellular crystallin modification or damage, the short-range order is disrupted and the cytoplasm exists as two separate phases. Under the microscope, the two phases have the appropriate size and refractive index difference to cause scattering of light. Light scattering is responsible for the opacity that cataract disease affects normal visual function. Typically, the Tc of lens proteins is well below body temperature under normal physiological conditions, so there is no light scattering and lens transparency is maintained. During cataract formation, Tc rises above body temperature, so at body temperature, crystallins phase separation occurs and the lens becomes cloudy.

Recent studies have shown that a large number of crystallins undergo post-translational modification, mutual aggregation and self-degradation, resulting in changes in the structure of crystallins, and the accumulation of these changes leads to the formation of cataracts. 90% of the proteins in the lens are structural proteins—crystallins, including three families of α, β and γ. The abnormal structure and function of crystal proteins and aggregation are important reasons for the formation of cataracts. However, the exact mechanism by which crystallins maintain transparency or develop opacity has not been fully understood until now.

For the drug treatment of cataract, the commonly used commercially available drugs are mainly divided into two categories: western medicine and traditional Chinese medicine. The western medicine includes: 1) aldose reductase inhibitors, such as catalin facolin, benzyl lysine, etc., 2) antioxidative damage drugs, such as glutathione, taurine, aspirin, etc., 3) nutritional metabolism drugs, such as vitamins, carotenoids, etc. At present, there is still an urgent clinical need to develop more effective pharmaceutical compositions for effectively treating cataracts and prevent the progression of cataracts, especially senile cataracts.

Since 2009, the understanding of phase separation proteins has gradually deepened, and a series of diseases related to phase separation have also been discovered. Many of these diseases have no effective clinical research methods and drugs. The present disclosure directly linked phase separation with disease treatment according to its pathogenic characteristics, which provides a new idea for the treatment of these diseases. The present disclosure provides use of polypeptides as a medicament to intervene in the physical state of proteins and achieve therapeutic effects, which provides the starting point of biophysical drugs.

In one aspect, the present disclosure provides a method for treating phase separation associated disease in a subject in need thereof, comprising administering to the subject in need thereof a therapeutically effective amount of a polypeptide that is capable of reversing phase separation, a polynucleotide encoding the polypeptide, and/or a vector comprising the polynucleotide. In certain embodiments, the phase separation associated disease is phase separation associated vision disorder, such as cataract. The term “cataract” as used herein refers to a disease or condition that shows symptoms, such as causing cloudiness or opacity on the surface and/or the inside of the lens, and/or inducing the swelling of the lens, which can be divided into congenital cataract and acquired cataract (see, PDR Staff “PDR of Ophthalmic Medicines 2013”, PDR Network, 2012). Exemplary congenital cataract includes but not limited to congenital pseudo-cataract, congenital membrane cataract, congenital lamellar cataract, congenital coronary cataract, congenital punctuate cataract, and congenital filamentary cataract. Exemplary acquired cataract includes but not limited to secondary cataract, geriatric cataract, browning cataract, complicated cataract, traumatic cataract, diabetic cataract, and others inducible by electric shock, radiation, drugs, systemic diseases, ultrasonic, and nutritional disorders. Exemplary acquired cataract can further include postoperative cataract with symptoms of causing cloudiness in the posterior encapsulating a lens inserted to treat cataract. In some embodiments, the cataract isa diabetic cataract, a cataract resulting from exposure to radiation, a cataract resulting from an infection, an age-related cataract, a cataract associated with surgery, a cataract resulting from a genetic illness, or a cataract resulting from medication.

In another aspect, the present disclosure provides a method for dissolving a protein aggregate (e.g., crystalline aggregate (e.g., TD crystallin aggregate), stress granule protein aggregate (G3BP1 protein aggregate)) and/or preventing the formation of crystalline protein aggregate (e.g., crystalline aggregate (e.g., TD crystallin aggregate), stress granule protein aggregate (G3BP1 protein aggregate)), comprising contacting the protein aggregate with a polypeptide that is capable of reversing phase separation.

In another aspect, the present disclosure provides a method for dissolving a protein aggregate (e.g., crystalline aggregate (e.g., TD crystallin aggregate), stress granule protein aggregate (G3BP1 protein aggregate)) and/or preventing the formation of crystalline protein aggregate (e.g., crystalline aggregate (e.g., TD crystallin aggregate), stress granule protein aggregate (G3BP1 protein aggregate)) in a cell, comprising introducing to the cell a polypeptide that is capable of reversing phase separation.

### Polypeptide, Polynucleotide, Vector

In some embodiments, the polypeptide comprises one or more of the following characteristics: 1) having a charge of greater than or equal to 30%, 2) having hydrophobic amino acids of 14%-26%, and polar amino acids of greater than or equal to 10%, and 3) the length is 20-100 amino acids. It should be understood that the charged amino acids can be replaced with each other, such as E mutated to D, or K, or R, etc.

In some embodiments, the polypeptide comprises a hydrophilic segment and a hydrophobic segment, said hydrophilic segment having a length of 10-20 (e.g., 11-19, 12-18, 13-17, 14-16, or 15) amino acid residues among which at least 50% (e.g., at least 50%, at least 55%, at least 60%, at least 65%, at least 70%, at least 75%, or at least 80%) are Asp, Glu, Lys, or Arg, said hydrophobic segment having a length of 10-20 amino acid residues among which at least 30% (e.g., at least 35%, at least 40%, at least 45%, at least 50%, at least 55%, at least 60%, at least 65%, at least 70%, at least 75%, or at least 80%) are Tyr, Phe, Trp, Leu, Ile, Val, Met, Pro, Ala, or Cys, wherein the hydrophilic segment is at the N-terminus and the hydrophobic segment is at the C-terminus, or vice versa, and wherein the polypeptide has a length of 20-60 (e.g., 25-55, 30-50, 35-45, or 40) amino acid residues.

In some embodiments, the hydrophilic segment has a sequence of selected from the group consisting of TX₁PQX₁X₁SX₁X₁X₁VX₁X₁PX₁X₁R (SEQ ID NO: 11), X₁LX₁X₁X₁SX₁X₁X₁VX₁X₁X₁QX₁X₁X₁ (SEQ ID NO: 12), X₁X₁X₁VX₁X₁X₁X₁X₁VX₁X₁ (SEQ ID NO: 13), and X₁X₁SX₁VQX₁LX₁ (SEQ ID NO: 14), wherein each X₁ is respectively Asp, Glu, Lys or Arg.

In some embodiments, the hydrophilic segment has a sequence selected from the group consisting of TEPQEESEEEVEEPEER (SEQ ID NO: 15), TDPQDDSDDDVDDPDDR (SEQ ID NO: 16), TKPQKKSKKKVKKPKKR (SEQ ID NO: 17), TRPQRRSRRRVRRPRRR (SEQ ID NO: 18), ELDEESEDEVEEEQEDR (SEQ ID NO: 19), KEEVDEDRDVDE (SEQ ID NO: 20), and EKSEQDLE (SEQ ID NO: 21), or a sequence having at least 90% identity thereto, or a sequence having 1, 2, 3, 4, or 5 amino acid residue difference therefrom.

In some embodiments, the hydrophobic segment has a sequence selected from: TFYDQTVSNDL (SEQ ID NO: 22), ANSAYYDAHPVTNGI (SEQ ID NO: 23), PPQTAAREATSIPGFPAEGAIPLPV (SEQ ID NO: 24), and EGEVAEEPNSRP (SEQ ID NO: 25), or a sequence having at least 90% identity thereto, or a sequence having 1, 2, 3, 4, or 5 amino acid residue difference therefrom.

In some embodiments, the polypeptide comprises a sequence selected from the group consisting of TEPQEESEEEVEEPEERQQTPEVVPDDSGTFYDQTVSNDLE (SEQ ID NO:1) (RJK001), TDPQDDSDDDVDDPDDRQQTPDVVPDDSGTFYDQTVSNDLD (SEQ ID NO:2) (RJK002), TKPQKKSKKKVKKPKKRQQTPKVVPDDSGTFYDQTVSNDLK (SEQ ID NO:3) (RJK012), TRPQRRSRRRVRRPRRRQQTPRVVPDDSGTFYDQTVSNDLR (SEQ ID NO:4), ELDEESEDEVEEEQEDRQPSPEPVQENANSAYYDAHIPVTNGIE (SEQ ID NO:
8), KEEVDEDRDVDESSPQDSPPSKASPAQDGRPPQTAAREATSIPGFPAEGAIPLPV (SEQ ID NO: 9), and EGEVAEEPNSRPQEKSEQDLE (SEQ ID NO: 10), or a sequence having at least 90% identity thereto, or a sequence having 1, 2, 3, 4, or 5 amino acid residue difference therefrom.

In some embodiments, the polypeptide comprises a hydrophilic segment and a hydrophobic segment, wherein the hydrophilic segment has a length of 10-17 (e.g., 11-16, 12-15, or 13-14) amino acid residues among which at least 60% (e.g., at least 65%, at least 70%, at least 75%, or at least 80%) are Asp, Glu, Lys, or Arg, wherein the hydrophobic segment having a length of 10-12 amino acid residues among which at least 35% (e.g., at at least 40%, at least 45%, at least 50%, at least 55%, at least 60%, at least 65%, at least 70%, at least 75%, or at least 80%) are Tyr, Phe, Leu, or Val, wherein the hydrophilic segment is at the N-terminus and the hydrophobic segment is at the C-terminus, or vice versa, wherein the polypeptide has a length of 20-29 (e.g., 21-28, 22-27, 23-26, or 24-25) amino acid residues, and wherein the polypeptide is capable of reversing phase separation.

In certain embodiments, the hydrophilic segment has a sequence of: TX₁PQX₁X₁SX₁X₁X₁VX₁X₁PX₁X₁R (SEQ ID NO: 11), wherein each X₁ is Asp, Glu or Lys.

In certain embodiments, the hydrophobic segment has a sequence of: TFYDQTVSNDL (SEQ ID NO: 22), or a sequence having at least 90% identity thereto, or a sequence having 1, 2, 3, 4, or 5 amino acid residue difference therefrom.

In certain embodiments, the polypeptide comprises a sequence of: TX₁PQX₁X₁SX₁X₁X₁VX₁X₁PX₁X₁RQQTPX₁VVPDDSGTFYDQTVSNDLX₁, wherein each X₁ is Asp, Glu or Lys.

It should be understood that hydrophilic amino acids other than Asp, Glu and Lys, such as Arg, Asn, Gln, His, Ser and Thr, may also be used as X₁ in the sequences mentioned above, and similar technical effect can be expected.

In some embodiments, the polypeptide comprises a sequence selected from the group consisting of: TEPQEESEEEVEEPEERQQTPEVVPDDSGTFYDQTVSNDLE (SEQ ID NO:1), TDPQDDSDDDVDDPDDRQQTPDVVPDDSGTFYDQTVSNDLD (SEQ ID NO:2), TKPQKKSKKKVKKPKKRQQTPKVVPDDSGTFYDQTVSNDLK (SEQ ID NO:3).

The polypeptides provided herein can be identified using the method described in PCT/CN2021/111683, disclosure of which has been incorporated herein by reference in its entirety.

In some embodiments, the polypeptide is fused with a cell-penetrating peptide (CPP). In some embodiments, the cell-penetrating peptide comprises a sequence selected from the group consisting of: GGRKKRRQRRR (SEQ ID NO: 26), and RQIKIWFQNRRMKWKKK (SEQ ID NO: 27). Other available CPP that can facilitate the cell entrance of the polypeptides provided herein are also within the contemplation of the present disclosure. The CPP can be fused to the N-terminal or C-terminal of the polypeptides provided herein.

In some embodiments, the polypeptide is further fused with a linker, for example, SGRPVL (SEQ ID NO: 28), GAPGSAGSAAGGSG (SEQ ID NO: 29), and/or ENLVFQG (SEQ ID NO: 30).

In some embodiments, the polypeptide is further fused with a his tag. The his tag used in the present disclosure could increase the half-life of the polypeptide provided herein.

In some embodiments, the polypeptide provided herein comprises a sequence of

(SEQ ID NO: 32)
TEPQEESEEEVEEPEERQQTPEVVPDDSGTFYDQTVSNDLEGGRKKRRQ
RRRHHHHHHHH
or
(SEQ ID NO: 33)
RQIKIWFQNRRMKWKKKTKPQKKSKKKVKKPKKRQQTPKVVPDDSGTFY
DQTVSNDLKSGRPVLHHHHHH.

The polypeptides provided herein can be produced by culturing a host cell (e.g., eukaryotic or prokaryotic cell) comprising the polynucleotides provided herein under a condition that allows expression of the polynucleotide provided herein. The polynucleotides provided herein can be constructed using Cell-free protein synthesis (CFPS) system. The polynucleotides provided herein can be constructed using recombinant techniques. To this end, DNA encoding the polynucleotide provided herein and DNA encoding the CPP, linker, and/or his tag can be obtained and operably linked to allow transcription and expression in a host cell to produce the fusion polypeptide.

For production of the fusion polypeptide provided herein, the host cells transformed with the expression vector may be cultured in a variety of media. Commercially available bacteria growth media such as Terrific Broth, LB Broth, LB Agar, M9 minimal media, MagiaMedia Medium, and ImMedia Medium (ThermoFisher) are suitable for culturing the bacterial host cells. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the eukaryotic host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In certain embodiments, the method further comprises isolating the fusion polypeptide and/or polypeptide complex.

The fusion polypeptide provided herein prepared from the cells can be purified using, for example, hydroxylapatite chromatography, gel electrophoresis, dialysis, DEAE-cellulose ion exchange chromatography, ammonium sulfate precipitation, salting out, and affinity chromatography.

Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein to be recovered.

In some embodiments, the polynucleotide is a DNA or an RNA. In some embodiments, the polynucleotide is single strand DNA or double strand DNA.

In another aspect, the present disclosure provides a host cell comprising the vector provided herein. The host cell is prokaryotic cell or a eukaryotic cell. Host cells transformed with the above-described expression or cloning vectors can be cultured in conventional nutrient media modified as appropriate for inducing promoters, selecting transformants, or amplifying the cloning vectors.

In some embodiments, the vector is a virus vector.

In some embodiments, the vector further comprises additional elements that facilitate the expression of the polypeptide, such as promoter, enhancer, polyA region, etc. In some embodiments, the vector is a virus vector. In some embodiments, the vector is an adeno-associated virus (AAV) vector. In some embodiments, the AAV vector further comprises an ITR sequence.

In some embodiments, the AAV has a serotype selected from AAV1, AAV2, AAV3, AAV5, AAV6, AAV7, AAV8, AAVrh8, AAV9, AAV10, AAVrh10, AAV11 and AAV12.

### Ophthalmic Pharmaceutical Composition

In another aspect, the present disclosure provides an ophthalmic pharmaceutical composition for treating phase separation associated visual disorder (e.g., cataracts) in a subject in need thereof, comprising a pharmaceutically acceptable ophthalmic carrier and a therapeutically effective amount of a polypeptide provided herein, a polynucleotide encoding the polypeptide, and/or a vector comprising the polynucleotide.

As used herein, “pharmaceutically acceptable ophthalmic carrier” refers to a pharmaceutically acceptable excipient, binder, carrier, and/or diluent for delivery of the polypeptide provided herein, the polynucleotide encoding the polypeptide, and/or the vector comprising the polynucleotide directly or indirectly to, on or near the eye.

The ophthalmic pharmaceutical composition can be in form of eye drops, which can be prepared using aqueous solutions and diluents, include but not limited to distilled water, physiological saline, and the like. Various additives may be contained in eye drops as needed. These can include but not limited to additional ingredients, additives or carrier suitable for use in contact on or around the eye without undue toxicity, incompatibility, soothing agents, instability, irritation, isotonicity adjusting agents, allergic response, and the like. Additives such as solvents, bases, solution adjuvants, suspending agents, thickening agents, emulsifying agents, stabilizing agents, buffering agents, pH-adjusting agents, flavoring agents, chelating agents, preservatives, corrigents, coloring agents, excipients, binding agents, lubricants, surfactants, absorption-promoting agents, dispersing agents, preservatives, solubilizing agents, and the like, can be added to a formulation where appropriate.

In one aspect, the present disclosure provides a method for treating phase separation associated visual disorder (e.g., cataracts) in a subject in need thereof, comprising administering to the subject in need thereof a therapeutically effective amount of the ophthalmic pharmaceutical composition described herein.

### Dosage

The therapeutically effective amount of the ophthalmic pharmaceutical composition provided herein will depend on various factors known in the art, such as for example type of disease to be treated, body weight, age, past medical history, present medications, state of health of the subject, immune condition and potential for cross-reaction, allergies, sensitivities and adverse side-effects, as well as the administration route and the type, the severity and development of the disease and the discretion of the attending physician or veterinarian. In certain embodiments, the pharmaceutical composition provided herein may be administered at a therapeutically effective dosage of about 0.001 mg/kg to about 100 mg/kg one or more times per day (e.g., about 0.001 mg/kg, about 0.3 mg/kg, about 0.5 mg/kg, about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg one or more times per day). In certain embodiments, the pharmaceutical composition is administered at a dosage of about 50 mg/kg or less, and in certain embodiments the dosage is 20 mg/kg or less, 10 mg/kg or less, 3 mg/kg or less, 1 mg/kg or less, 0.3 mg/kg or less, 0.1 mg/kg or less, or 0.01 mg/kg or less, or 0.001 mg/kg or less. In certain embodiments, the administration dosage may change over the course of treatment. For example, in certain embodiments the initial administration dosage may be higher than the subsequent administration dosages. In certain embodiments, the administration dosage may vary over the course of treatment depending on the reaction of the subject.

Dosage regimens may be adjusted to provide the optimum desired response (e.g., a therapeutic response). In certain embodiments, the ophthalmic pharmaceutical composition provided herein is administered to the subject at one time or over a series of treatments. In certain embodiments, the pharmaceutical composition provided herein is administered to the subject by one or more separate administrations, or by continuous infusion depending on the type and severity of the disease.

The ophthalmic pharmaceutical compositions provided herein can be administered as a single dose or in multiple doses. The ophthalmic pharmaceutical compositions provided herein can be administered either as individual therapeutic agents or in combination with other therapeutic agents, or combined with conventional therapies, which can be administered sequentially or simultaneously. In some embodiment, the ophthalmic pharmaceutical compositions provided herein is administered at a daily dosage of about 1 drop per eye, about 2 drops per eye, about 3 drops per eye, about 4 drops per eye, about 5 drops per eye, about 6 drops per eye, about 7 drops per eye, about 8 drops per eye, about 9 drops per eye, about 10 drops per eye, about 11 drops per eye, about 12 drops per eye or more than about 12 drops per eye. In some embodiments, the ophthalmic pharmaceutical compositions provided herein is administered about 1 time per day, about 2 times per day, about 3 times per day, about 4 times per day, about 5 times per day, about 6 times per day, about 7 times per day, about 8 times per day, about 9 times per day, about 10 times per day, about 11 times per day, about 12 times per day or more than about 12 times per day.

### Route of Administration

The most appropriate method of administering the polypeptide provided herein, the polynucleotide provided herein, the vector provided herein and/or the pharmaceutical composition provided herein to a subject is dependent on a number of factors. In various embodiments, the polypeptide provided herein, the polynucleotide provided herein, the vector provided herein and/or the pharmaceutical composition provided herein is administered locally to the eye, e.g., topically, subconjunctivally, retrobulbarly, periocularly, subretinally, suprachoroidally, or intraocularly.

The pharmaceutical compositions provided herein are formulated such that they are particularly useful for administration directly to the eye. Exemplary formulations for the pharmaceutical compositions provided herein include but not limited to eye drops (which are in forms of aqueous solutions and/or suspensions), ophthalmic gels or ointments (which are in forms of thickened solutions and/or suspensions), ophthalmic wash, wash for anterior chamber, intraocular infusion solution, internal medicine, or preservative for extracted cornea.

Other dosage forms for ophthalmic drug deliver include ocular inserts, intravitreal injections and implants. Injectable solutions can be directly injected into the cornea, crystalline lens and vitreous or their adjacent tissues using a fine needle. The composition also can be administered as an intraocular perfusate.

In some embodiments, the administration route is via a contact lens. The lens may be provided pre-treated with the polypeptide provided herein, the polynucleotide provided herein, the vector provided herein and/or the pharmaceutical composition provided herein. In an alternative embodiments, the lens is provided in a kit with components for preparing a coated lens, which are provided as lyophilized powders for reconstitution or as concentrated or ready-to-use solutions. The compositions can be provided as kits for single or multi-use.

### Pharmaceutical Composition for Gene Therapy

In certain embodiments, the polynucleotide and/or the vector provided herein can be formulated as a pharmaceutical composition that be injected directly to an eye of a subject in need thereof for gene therapy. Such pharmaceutical composition can further comprise a pharmaceutically acceptable vehicle. In some embodiments, the pharmaceutically acceptable vehicle is a liposome. Liposomes are monolayer or multilayer vesicles with membranes formed by lipophilic materials and an internal aqueous portion. The polypeptide and/or vector provided herein can be encapsulated in the aqueous portion of the liposome. Exemplary liposomes comprise, but are not limited to, liposomes based on 3[N-(N′,N′-dimethylaminoethane) carbamoyl]cholesterol (DC-Chlo), liposomes based on N-(2,3-dioleoxy)propyl-N,N,N-trimethyl ammonium chloride (DOTMA), and liposomes based on 1,2-dioleoyl-3-trimethylpropane (DOTAP). Methods of preparing liposomes and encapsulating nucleic acid molecules and/or vectors into liposomes are well known in the art. (See, e.g., D. D. Lasic et al, Liposomes in gene delivery, published by CRC Press, 1997).

In certain embodiments, the pharmaceutically acceptable vehicle is a polymeric excipient, including but not limited to microspheres, microcapsules, polymeric micelles and dendrimers. Nucleic acid molecules and/or vectors provided herein can be encapsulated, adhered to, or coated on polymer-based components by methods known in the art (See, e.g., W. Heiser, Nonviral gene transfer techniques, published by Humana Press, 2004; U.S. Pat. No. 6,025,337; Advanced Drug Delivery Reviews, 57(15): 2177-2202 (2005)).

In certain embodiments, the pharmaceutically acceptable vehicle is colloid or vector particles, such as gold colloids, gold nanoparticles, silica nanoparticles and multi-segment nanorods. Nucleic acid molecules and/or vectors provided herein can be coated on, adhered to or bound to vectors by any suitable method known in the art (See, e.g., M. Sullivan et al., Gene Therapy, 10: 1882-1890 (2003), C. McIntosh et al., J. Am. Chem. Soc., 123 (31): 7626-7629 (2001), D. Luo et al., Nature Biotechnology, 18: 893-895 (2000), and A. Salem et al., Nature Materials, 2: 668-671 (2003)).

In certain embodiments, pharmaceutical composition can further comprise additives, including, but not limited to, stabilizers, preservatives, and transfection promoters that facilitate cellular uptake of the drug. Suitable stabilizers can include, but are not limited to, sodium glutamate, glycine, EDTA and albumin. Suitable preservatives can include, but are not limited to, 2-phenoxyethanol, sodium benzoate, potassium sorbate, methyl hydroxybenzoate, phenols, thiomersal and antibiotics. Suitable transfection promoters can include, but are not limited to, calcium ions.

The pharmaceutical composition provided herein can be administered by any suitable route known in the art, including, but not limited to, the gastrointestinal, oral, intestinal, buccal, nasal, local, rectal, vaginal, intramuscular, intranasal, mucosal, epidermal, transdermal, dermal, ocular, pulmonary, intravitreal injection, anterior chamber injection, subarachnoid injection and subcutaneous routes. The pharmaceutical composition provided herein can be administered to subjects in preparations or forms of formulations suitable for each route of administration. Formulations suitable for administration of pharmaceutical composition can include, but are not limited to, solutions, dispersions, emulsions, powders, suspensions, aerosols, sprays, nasal drops, liposomal based formulations, patches, implants, and suppositories.

Formulations can be easily present in unit dosage forms and can be prepared by any method known in the pharmaceutical field. Methods of preparing these formulations or pharmaceutical compositions include steps for supplying the polynucleotides described herein to one or more pharmaceutically acceptable vehicles and optionally one or more additives. For the methods of preparing such formulations, see, e.g., Remington's Pharmaceutical Sciences (Remington: The Science and Practice of Pharmacy, 19th ed., A. R. Gennaro (ed), Mack Publishing Co., NJ, 1995; R. Stribling et al., Proc. Natl. Acad. Sci. USA, 89:11277-11281 (1992); A. Barnes et al., Current Opinion in Molecular Therapeutics 2000 2:87-93(2000); T. W. Kim et al., The Journal of Gene Medicine, 7(6): 749-758(2005); and S. F. Jia et al., Clinical Cancer Research, 9:3462 (2003); A. Shahiwala et al., Recent patents on drug delivery and formulation, 1:1-9 (2007); the references are incorporated herein by reference in their entirety).

In some embodiments, the polynucleotides provided herein (e.g. mRNA) may be delivered by physical, biological or chemical methods (see, e.g., S. Guan, J. Rosenecker, Gene Ther. 2017, 24, 133).

Physical methods include, but are not limited to, delivering via gene guns (e.g. gene guns with Au-particles), electroporation, acoustic perforation, etc (see, e.g., Kutzler et al., (2008) DNA vaccines: Ready for prime time?Nat Rev Genet 9: 776-788; Geall et al., Nonviral delivery of self-amplifying RNA vaccines. Proc Natl Acad Sci USA. Sep. 4, 2012; 109(36):14604-9).

Biological methods include, but are not limited to, delivering via viral vectors (e.g., retrovirus vectors, adenovirus vectors, adeno-associated virus vectors).

Chemical methods include, but are not limited to, delivering via natural proteins/glycans, polymers, lipids. Exemplary natural proteins/glycans include protamine and chitosan (see, e.g., A. E. Sköld et al., Cancer Immunol. Immunother. 2015, 64, 1461; U. S. Kumar et al. ACS Nano 2021, 11, 17582). Exemplary polymers include polyethylene imine (PEI) (such as, linear PEI, branched PEI and dendritic PEI), Poly(β-amino ester) (PBAE) (see, e.g., K. Singha et al., Nucleic Acid Ther. 2011, 21, 133; A. A. Eltoukhy et al., Biomaterials 2012, 33, 3594).

Exemplary lipids include cationic lipids, such as 1,2-diocta-decenyl-3-trimethylammonium-propane (DOTMA) and 1,2-dioleoyl-3-trimethylammoniumpropane (DOTAP) (see, e.g., X Hou et al., Nat. Rev. Mater. 2021, 10, 1078), liposomes formed by DOTMA, DOTAP and DOPE (1,2-dioleoyl-sn-glycero-3-phosphoethanolamine, auxiliary lipid), which can form colloidal stabilized nanoparticles after self-assembling with mRNA (see, e.g., L. M Kranz et al., Nature 2016, 534, 396).

Exemplary lipids may also be ionizable lipids. Ionizable lipid (pKa 6.5-6.9) is an alternative lipid material that is neutral at physiological pH but positively charged by protonation of free amines in an acidic environment (see, e.g., S. C. Semple et al., Nat. Biotechnol. 2010, 28, 172). After cell internalization, nanoparticles formed by ionizable lipids are encapsulated in endosomes. Subsequently, due to the continuous decrease of pH in endosomes and lysosomes, ionizable lipids gain protons for ionization, which promotes the fusion of lipid nanoparticle (LNP) with endosomal membrane, and finally leads to the release of mRNA loaded on lipid nanoparticles into the cytoplasm (see, e.g., L. Miao et al., Mol. Cancer 2021, 20, 41).

Ionizable lipids can form lipid nanoparticle formulations with cholesterol, auxiliary lipids, and pegylated lipids (i.e., PEGylated lipids). Cholesterol is a naturally rigid and hydrophobic lipid that maintains the structure and stability of lipid nanoparticles. It also promotes the fusion of mRNA-loaded lipid nanoparticles (i.e., mRNA nanoparticles) with the endosomal membranes. Auxiliary lipids (e.g., zwitterionic lipid DOPE, 1,2-distearoyl-snglycero-3-phosphocholine (DSPC) and 1,2-Dioleoyl-sn-glycero-3-phosphocholine (DOPC)) are widely used to promote cell membrane penetration and endosomal membrane escape (see, e.g., N. Chaudhary et al., Nat. Rev. Drug Discovery 2021, 20, 817). PEGylated lipids are composed of PEG and anchored lipids. Hydrophilic PEG is mainly distributed on the surface of the mRNA complex, while the hydrophobic region is embedded in the lipid bilayer. The introduction of PEGylated lipids not only increases the half-life of lipid nanoparticles but also can adjust the particle size by changing the molecular weight of the PEG chain. Typically, molecular weight and lipid tail length can range from 350 to 3000 Da and 10 to 18 carbons, respectively (see, e.g., N. Chaudhary et al., Nat. Rev. Drug Discovery 2021, 20, 817).

Over the past few decades, researchers have developed large libraries of ionizable lipids for mRNA delivery, including DLin-MC3-DMA, SM-102, TT3, C12-200, 306Oᵢ₁₀ and ALC-0315 (see, e.g., M. Yanez Arteta et al., Proc. Natl. Acad. Sci. USA 2018, 115, E3351; R. Verbeke et al., Controlled Release 2021, 333, 511; B. Li et al., Nano Lett. 2015, 15, 8099; K. A. Hajj et al., Nano Lett. 2020, 20, 5167; K. A. Hajj et al., Small 2019, 15, 1805097; A. B. Vogel et al., Nature 2021, 592, 283). Some of them have achieved remarkable results in clinical application. A typical example is DLin-MC3-DMA, a key component of Onpattro approved by the US Food and Drug Administration (FDA) for siRNA delivery (see, e.g., A. Akinc et al., Nat. Nanotechnol. 2019, 14, 1084). DLin-MC3-DMA is also widely used for mRNA delivery, including protein and peptide substitution, gene editing, and antiviral infections (see, e.g., R. S. Riley et al., Sci. Adv. 2021, 7, eaba1028). Two “star molecules”, SM-102 and ALC-0315, have been approved by the FDA as key components in BNT162b and mRNA-1273 vaccines for preventing COVID-19, respectively (see, e.g., X Hou et al., Nat. Rev. Mater. 2021, 10, 1078).

The ideal lipid-based mRNA vector must meet the following requirements: 1) the exposed mRNA can form a stable complex that protects the mRNA from degradation; 2) four key components (ionizable lipids, cholesterol, auxiliary lipids, and pegylated lipids) should be added to stabilize the mRNA complex; 3) the composition of lipid nanoparticles should be protonated to trigger membrane instability and promote endosomal escape of the mRNA complex; and 4) all lipid materials are biodegradable and will not cause any harm to the patients.

The following points should be considered when optimizing lipid-based delivery platforms: 1) the ability of degradation of ionizable lipids: the skeleton structure of lipid facilitates clearance of lipid and reduces toxicity by introducing alkyne and ester groups into the lipid tail; 2) immunogenicity of lipid nanoparticles: the heterocyclic lipids in lipid nanoparticles can improve the efficiency of mRNA vaccines by activating the pathway of stimulator of interferon gene (STING) of dendritic cells (DCS) (see, e.g., L. Miao et al., Nat. Biotechnol. 2019, 37, 1174); 3) stability of lipid nanoparticles: some potential strategies for improving stability of mRNA vaccines include optimization of pKa, introduction of excipient, and modification of mRNA, etc.

Lipid nanoparticles can be produced using components, compositions and methods known in the art, see, e.g. PCT/US2016/052352, PCT/US2016/068300, PCT/US2017/037551, PCT/US2015/027400, PCT/US2016/047406, PCT/US2016/000129, PCT/US2016/014280, PCT/US2016/014280, PCT/US2017/038426, PCT/US2014/027077, PCT/US2014/055394, PCT/US2016/052117, PCT/US2012/069610, PCT/US2017/027492, PCT/US2016/059575 and PCT/US2016/069491, which are incorporated herein by reference in their entirety.

### Kits

In another aspect, the present disclosure provides a kit for treating phase separation associated diseases (e.g., cataracts), comprising a formulation of a therapeutically effective amount of the polypeptide provided herein, the polynucleotide provided herein, and/or the vector provided herein, a pharmaceutically acceptable carrier and instructions for administering the formulation such that the administration treats the phase separation associated diseases.

In some embodiments, the kits comprise one or more containers that contain one or more of the polypeptide provided herein, the polynucleotide provided herein, and/or the vector provided herein. The polypeptide provided herein, the polynucleotide provided herein, and/or the vector provided herein can be present in the container as a prepared pharmaceutical composition, or alternatively, can be unformulated. In such embodiments, the kit can include the polypeptide provided herein, the polynucleotide provided herein, and/or the vector provided herein that is unformulated in a container, which is separate from the pharmaceutically acceptable carrier that is present in another container. Prior to use, the polypeptide provided herein, the polynucleotide provided herein, and/or the vector provided herein in diluted or otherwise mixed with the pharmaceutically acceptable carrier.

In some embodiment, the kits provided herein also comprise instructions that describe the method for administering the pharmaceutical composition in such a way that one or more symptoms associated with phase separation associated diseases (e.g., cataracts). In some embodiments, the instructions also describe the procedure for mixing the polypeptide provided herein, the polynucleotide provided herein, and/or the vector provided herein contained in the kit with ophthalmic pharmaceutically acceptable carriers.

In some embodiments, the container that comprises the polypeptide provided herein, the polynucleotide provided herein, and/or the vector provided herein is a container which is used for ophthalmic administration. In some embodiments, the container is a dropper for administering eye drops.

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### V. EXAMPLES

### Example 1: Phase Separation Reversion on G3BP1 Proteins

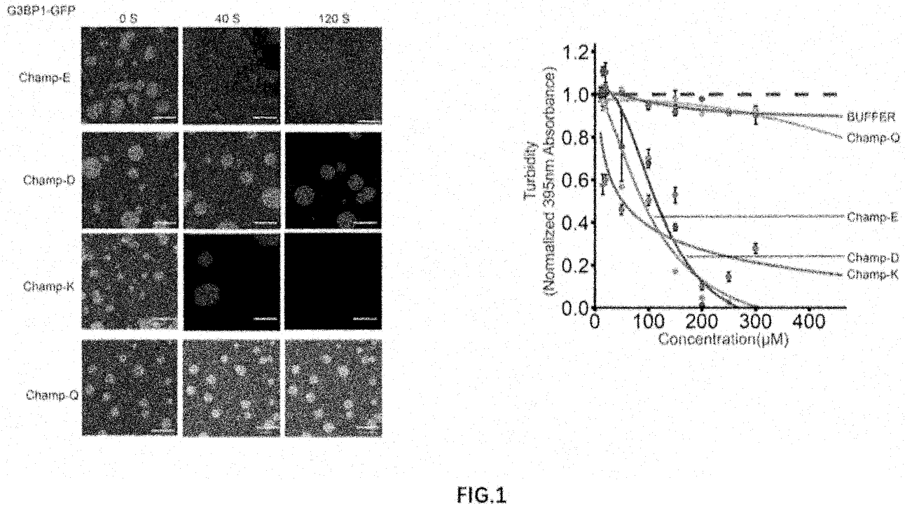

The prokaryotic expressed and purified G3BP1-GFP protein (pH7.5, 20 uM G3BP1-GFP) was induced to form phase separated droplets in vitro. Polypeptides Champ-E (RJK001), Champ-D (RJK002), Champ-K (RJK012) and Champ-Q (RJK005, TQPQQQSQQQVQQPQQRQQTPQVVPDDSGTFYDQTVSNDLQ, SEQ ID NO: 34) (each of 60 uM) were added dropwise to the G3BP1-GFP droplets. The observation was performed continuously through a confocal laser microscope. As shown in FIG. 1 left panel, wherein the left image is the microscopic image just after adding the polypeptides, the middle image is the microscopic image after the polypeptides were added for 40 seconds, and the right image is the microscopic image after the polypeptides were added for 80 seconds, the polypeptides Champ-E and Champ-K significantly dissolved the phase separated droplets at 40s after the addition of the polypeptides, Champ-D partially dissolved the phase separated droplets at 40s after the addition of the polypeptides and dissolved more phase separated droplets at 120s after the addition of the polypeptides, whereas Champ-Q did not dissolve the phase separated droplets even at 120s after the addition of the polypeptides. This suggested that the polypeptides Champ-E (RJK001), Champ-D (RJK002) and Champ-K (RJK012) could reverse phase separation in a time-dependent manner.

The turbidity of G3BP1 (30 μM) in the presence of various concentrations of Champ-E, Champ-D, Champ-K, or Champ-Q was measured at a wavelength of 395 nm. Briefly, G3BP1 protein (30 M) and polypeptides Champ-E (RJK001), Champ-D (RJK002), Champ-K (RJK012) and Champ-Q (RJK005) were mixed well in a ratio range of 0.75 to 25 at room temperature for 10 minutes prior to turbidity assays. Absorbance at 395 nm (OD395 nm) was monitored at room temperature using Nanodrop ONE (Thermo Fisher Scientific, Inc.). As shown in FIG. 1 right panel, polypeptides Champ-E, Champ-D and Champ-K reduced the turbidity in a concentration dependent manner, whereas Champ-Q had much less effect on the reduction of the turbidity. The turbidity level reflects the degree of phase separation. Hence, this suggested that polypeptides provided herein (e.g., Champ-E, Champ-D and Champ-K) could reserve phase separation in a dose-dependent manner.

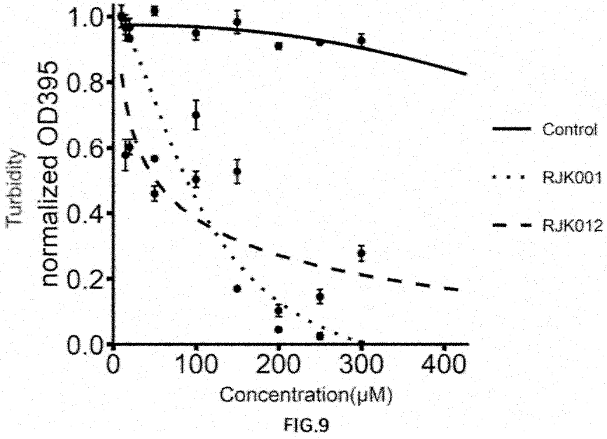

The turbidity of G3BP1 (30 μM) in the presence of various concentrations of RJK001 fused at its C-terminal with a cell-penetrating peptide (GGRKKRRQRRR) and RJK012 fused at its N-terminal with a cell-penetrating peptide (RQIKIWFQNRRMKWKKK) was measured at a wavelength of 395 nm. Briefly, the G3BP1 protein and the above-mentioned peptides were mixed at room temperature for 10 minutes prior to turbidity assays. 30 μM G3BP1 and peptide were mixed well in a ratio range of 0.75 to 25 before test. Absorbance at 395 nm (OD395 nm) was monitored at room temperature using Nanodrop ONE (Thermo Fisher Scientific, Inc.). As shown in FIG. 9, both the polypeptide RJK001 and RJK012 each fused with a cell-penetrating peptide either at N-terminal or C-terminal could reduce the turbidity in a concentration dependent manner (The “RJK001” and “RJK012” in FIG. 9 each has a cell-penetrating peptide fused at the C terminus or N terminus). This suggested that the polypeptides provided herein fused with a cell-penetrating peptide either at N-terminal or C-terminal (e.g., RJK001 and RJK012) could reserve phase separation in a dose-dependent manner. This result further suggested that fusing with a cell-penetrating peptide did not affect the capability of the polypeptides provided herein for dissolving or reversing phase separation.

### Example 2: Phase Separation Reversion on γD-crystallin Proteins in Tubes

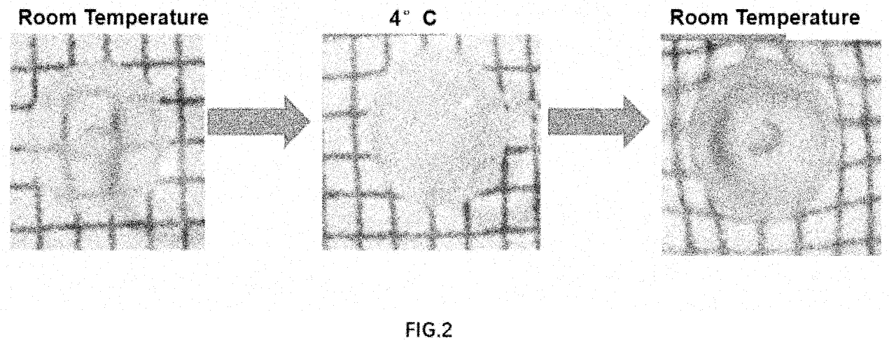

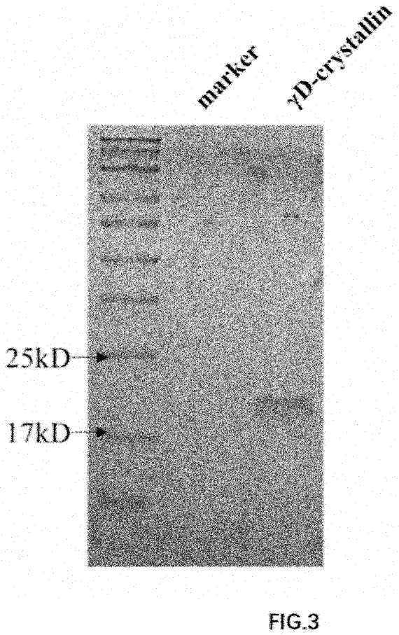

A mouse lens was placed at different temperatures and photographs were taken. As shown in FIG. 2, the mouse lens showed clarity at room temperature, which then became cloudy at 4° C., and came back to clarity after the temperature increased to room temperature. This suggested that γD-crystallin protein exhibited phase separation at low temperature (e.g., 4° C.), which was reversible when the temperature increased to room temperature. Next, the inventors of the present disclosure tested whether the polypeptides provided herein could reverse the phase separation of γD-crystallin, thereby treating visual disorders induced by phase separation of γD-crystallin, such as cataracts.

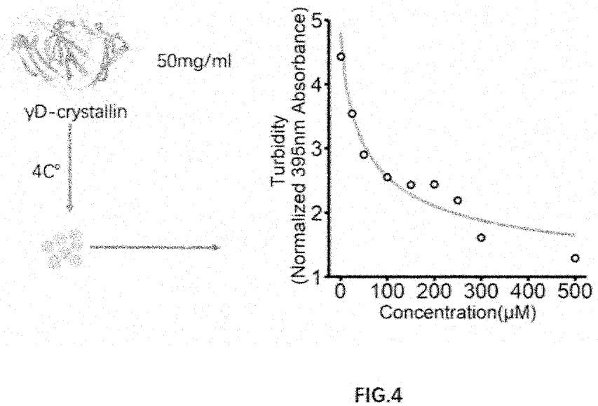

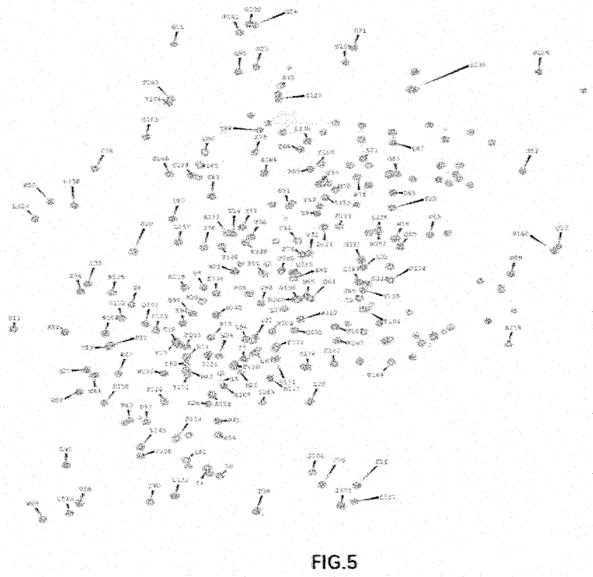

γD-crystallin was purified in vitro, which was validated as shown in FIG. 3. The purified γD-crystallin was then dissolved in protein solubilization buffer to a final concentration of 50 mg/ml, Place γD-crystallin at 4° C. for 20 minutes to form a stable biphasic phase, add different concentration of the short peptide RJK001 in Sul γD-crystallin in a volume ratio of 1:1, final concentration of RJK001 from low to high is 0 uM, 20 uM, 50 uM, 100 uM, 150 uM, 200 uM, 250 uM, 300 uM and 500 uM, Use a 10 ul pipette tip to mix them well, then place them at 4° C. for 5 min, absorbance at 395 nm (OD395 nm) was monitored at at 4° C. using Nanodrop ONE (Thermo Fisher Scientific, Inc.). As shown in FIG. 4, polypeptide RJK001 reduced the turbidity of γD-crystallin in a concentration dependent manner. The turbidity level reflects the degree of phase separation. Hence, this suggested that polypeptides provided herein (e.g., RJK001) could reserve phase separation of γD-crystallin in a dose-dependent manner.

To evaluate whether a protein undergoes small or significant structural changes before and after adding polypeptides, ¹H-¹⁵N HSQC spectroscopy is a useful tool and can be used as a “fingerprint” of the three-dimensional structure. As shown in FIG. 5, we expressed the full-length γD-crystallin protein in E. coli, characterized it by a bruker avance III HD spectrometer. Wild-type human full-length γD-crystallin gene were inserted into pET14b vectors. E. coli BL21 (DE3) cells were transformed with these vectors. For protein production, cells were grown at 37° C. to an absorbance at 600 nm of 0.6 and induced by the addition of 0.5 mM IPTG at 37° C. for 4 h. For the N15 labeling of the protein, the transformed E. coli BL21(DE3) cells were cultured and induced in M9 medium with NH₄Cl as the only nitrogen source. Protein was purified by Ni affinity chromatography In Tris HCl pH 7.5, 150 mM NaCl using a linear gradient of imidazole. All the fractions containing γD-crystallin were pooled and subjected to gel filtration on Supdex75 column (GE Healthcare) for the final purification. The purified protein was loaded to SDS-PAGE for the purity check before HSQC spectroscopy assay. Using final 300 uM human γD-crystallin at 25° C., the two-dimensional [¹H-¹⁵N] heteronuclear single quantum coherence (HSQC) spectra were recorded to determine the domain interactions at the residue-specific level.

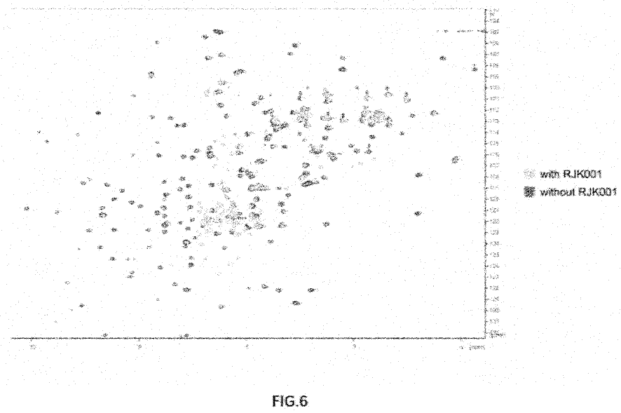

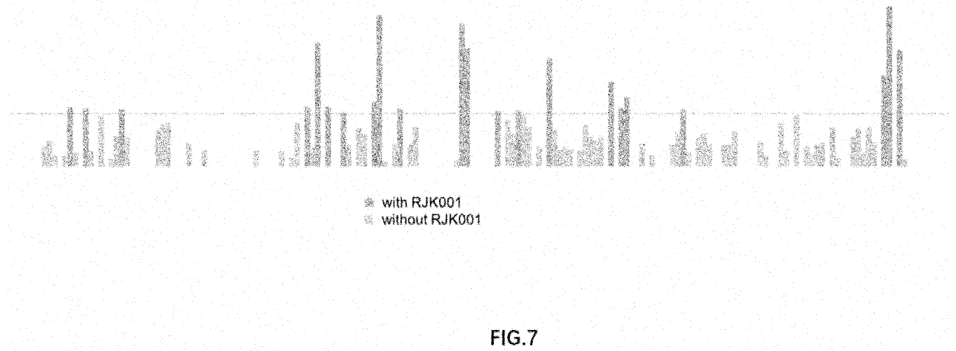

H-N analyses revealed a change in the environment of individual residues as indicated by their corresponding chemical shifts. Chemical shift difference (CSD) was measured for γD-crystallin alone or with Champ-E(RJK001). As shown in FIG. 6, we first recorded the signal 2D 1H-15N HSQC spectra of 300 M 15N γD-crystallin alone (dark grey), and then 1.2 mM RJK001 solution in the same volume was added into the crystallin solution to reach a molar ratio of 1:2 in final. After the addition of the 1.2 mM RJK001 to the 15N-labeled samples, ¹H-¹⁵N HSQC spectra were recorded as a function of time, we recorded the signal 2D ¹H-¹⁵N HSQC spectra of 300 M 15N γD-crystallin with RJK001 (light grey). Comparison of the 1H-15N HSQC spectra prior to and after RJK001 incubation shows that a significant chemical shifts, especially in polar amino acid regions.

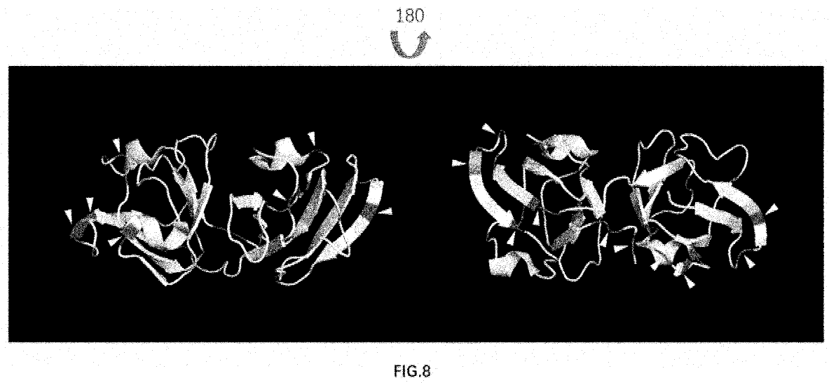

HSQC spectrums were acquired and analysis with ccpNMR, amide ¹H-¹⁵N combined chemical shift differences were calculated using the equation Δδ=[(0.125ΔδN)²+ΔδH²]^{1/2}. In the presence of RJK001 (final 600 mM), CSD of total residues of γD-crystallin was observed as shown in FIG. 7. CSD of the γD-crystallin showed greater change suggesting that RJK001 interacted with the full-length wildtype γD-crystallin protein, thereby tightening the surface tension and increasing γD-crystallin solubility.

As shown in FIG. 8, residues with ¹H, ¹⁵N chemical shift differences >0.1 ppm compared to the full-length wildtype γD-crystallin are labeled with arrows. The schematic diagram depicts the backbone structure of wildtype γD-crystallin (2KFB) onto which the chemical differences are mapped. Positions of residues whose amide resonances exhibit Δδ>0.1 ppm are shown with arrows, suggesting the location of RJK001 interaction. Indeed, all data available at present support the notion that heterotypic interactions between the different types of crystallins are delicately balanced in the eye lens, such that even slight strengthening or weakening of such interactions may lead to instability in mixtures of these proteins, 30 with a-crystallin, by virtue of its chaperone properties, playing a pivotal role in preventing the aggregation and/or precipitation of other lens crystallins. So RJK001 increased γD-crystallin solubility using such interactions.

### Example 3: Phase Separation Reversion on γD-crystallin Proteins in Cells

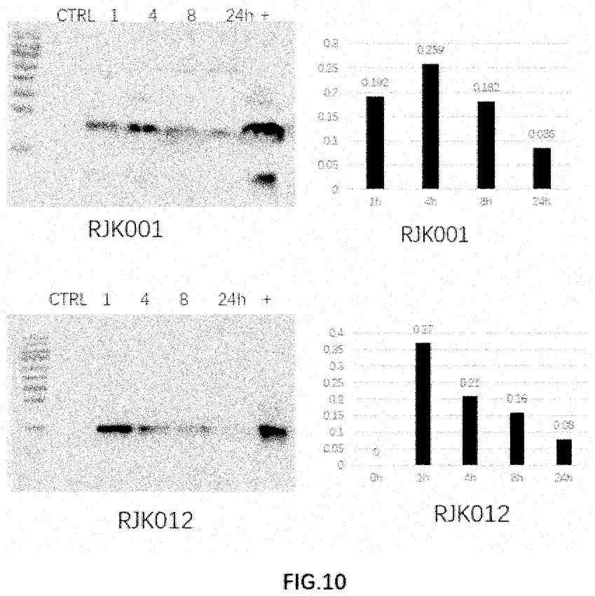

As described in Example 1, the polypeptide RJK001 was fused at its C-terminal with a cell-penetrating peptide (GGRKKRRQRRR) and the polypeptide RJK012 was fused at its N-terminal with a cell-penetrating peptide (RQIKIWFQNRRMKWKKK). The half-life of the fused peptides in eukaryotic cells were tested, and the results were shown in FIG. 10, which suggested that the half-life of the fusion polypeptides in eukaryotic cells were about 8 hours.

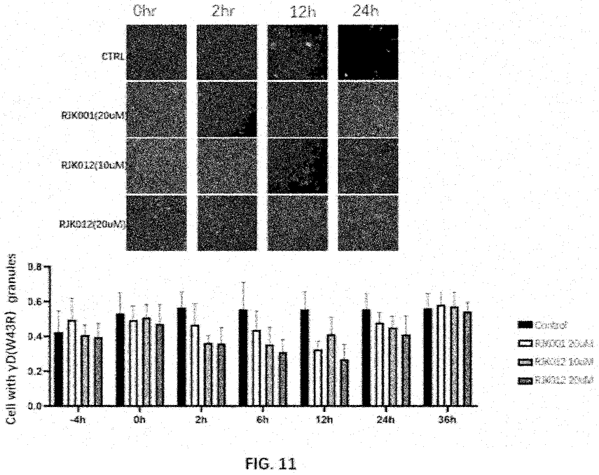

Cells were grown in a 12-well plate (1×10⁵ cells per well) for 24 hours and transfected with γD (W42D) plasmid using EZtrans transfection reagent. 24 hours after transfection, Champ peptides RJK001 (final concentration of 20 uM) and RJK0012 (final concentration of 10 uM and 20 uM) were added into SH-SY5Y cell culture medium every 4 hours for a total of 24 hours. The effect of RJK001 and RJK012 polypeptide on the γD (W42D) aggregates in the SH-SY5Y cell mode were analyzed using a custom CellProfiler software. Briefly, cell bodies were then segmented and identified by the GFP fluorescence channel images and tracing outward to the limits of the cytoplasmic (using a diameter cutoff of 100-300 pixels). The cell bodies were used as masks to eliminate imaging artifacts outside of cell boundaries, such as background fluorescence or dead cells. After masking, punctate structures were enhanced by image processing for speckle-like features that were 10 pixels in diameter for SH-SY5Y cells and these punctate structures were then annotated as features such as γD (W42D) puncta. Finally, the total image area which was enclosed in each of the identified features (number of cell and punctate) was calculated and output to spreadsheets. At least 200 cells per sample were analyzed. As shown in FIG. 11, the control group did not affect the γD (W42D) aggregates in the SH-SY5Y cell as time passed; the polypeptides RJK001 of 20 uM and RJK012 of 20 uM reduced the number of γD (W42D) aggregates in the SH-SY5Y cell in a time-dependent manner until 12 hours after addition of the polypeptide; and the polypeptide RJK012 of 10 uM reduced the number of γD (W42D) aggregates in the SH-SY5Y cell in a time-dependent manner until 6 hours after addition of the polypeptide. These data suggested that the polypeptides provided herein (e.g., RJK001 and RJK012) could dissolve or reverse the γD crystallin protein aggregates inside eukaryotic cells in a dose-dependent and time-dependent manner.

### Example 4: Treatment and Analysis of Human Lenses

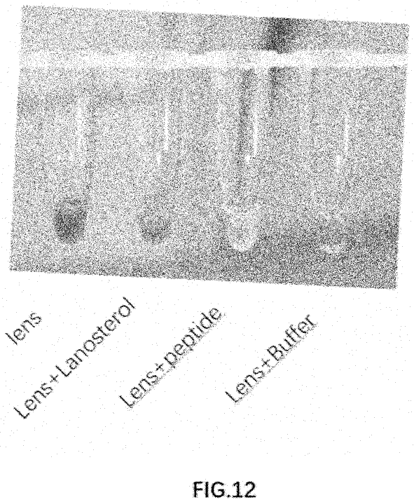

30-50 ml lens lysates after phacoemulsification was collected from cataract patients with different disease progressions. Lanosterol was used as a benchmark medicament (see, Zhao, Ling, et al. “Lanosterol reverses protein aggregation in cataracts.” Nature 523. 7562 (2015): 607-611). As shown in FIG. 12, the polypeptides provided herein (e.g., RJK001) could effectively dissolve the crystallin aggregation from cataract patients. Moreover, the concentration of the polypeptides provided herein (e.g., RJK001) was as less as 1/100 of the concentration of Lanosterol required to achieve the same dissolving effect. This result suggested that the polypeptides provided herein (e.g., RJK001) exhibited better dissolving effect on crystallin aggregation from cataract patients than Lanosterol.

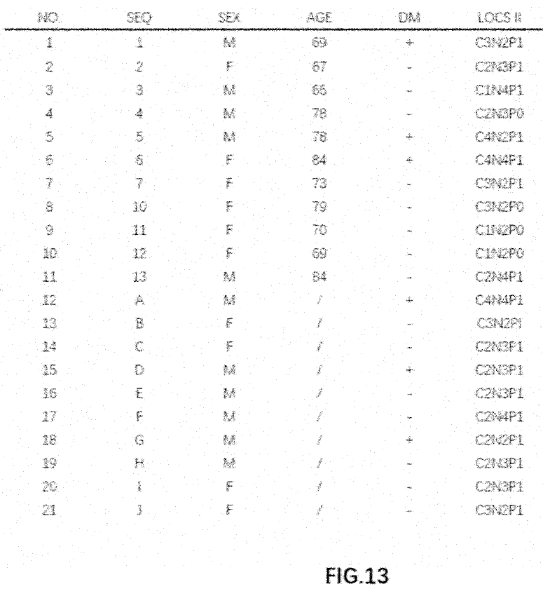

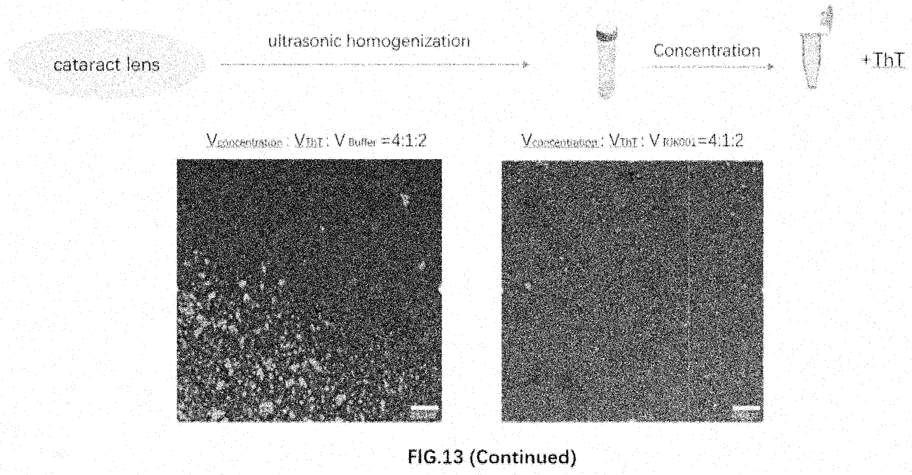

Human lenses used in this study were obtained from unidentified patients undergoing extracapsular cataract surgery. The material had been classified as non-human discarded material. Preoperative clinical exam was used to classify the cataract as mixed cortical and nuclear using LOCS II four-point grading system. LOCS II four-point grading system is also commonly used in clinical settings. (Chylack LT Jr, Leske MC, McCarthy D, Khu P, Kashiwagi T, Sperduto R. Lens opacities classification system II (LOCS II). Arch Ophthalmol. 1989 July; 107(7):991-7. doi: 10.1001/archopht.1989.01070020053028. PMID: 2751471). We tested the crystallin concentrates of patients with different degrees of cataract one by one. As shown in FIG. 13, the polypeptides provided herein (e.g., RJK001) had good depolymerization effects on patients with different degrees (e.g., C1N2P0) of cataract.

### Grading System of Human Cataracts.

N Grade 0: absence of opacification (no cataract);
N Grade 1: a slight degree of opacification (incipient stage);
N Grade 2: presence of diffuse opacification involving almost the entire lens (immature stage);
N Grade 3: presence of extensive, thick opacification involving the entire lens (mature stage)
C Grade 0: clear lens devoid of aggregated dots, flecks (no cataract);
C Grade 1: minimal degree of cortical opacification and/or more extensive opacification with small;
C Grade 2: cortical spoking that obscures more than 2 full;
C Grade 3: Opacification that obscures about 50% of the;
C Grade 4: Advanced opacification filling about 90% of the lens
P Grade 0: Clear posterior capsule (no cataract);
P Grade 1: Cataract filling about 3% of the area of the posterior capsule;
P Grade 2: About 30% opacification of the area of the posterior capsule;
P Grade 3: About 50% opacification of the area of the posterior capsule.

30-50 ml Phacoemulsification of eye lens was collected from cataract patients with different disease progression. Let Phacoemulsification pass the 3KD protein concentration tube, and centrifuge at 4000 g for 20 minutes. centrifuge at 14000 g for 5 minutes after repeating the previous step to get the concentrate. Concentrate phacoemulsification to 200-300 ul every lens. Add 8 ul phacoemulsification concentrate in the following groups: 1, 8 ul crystalline concentrate; 2, 8 ul 1 mM lanosterol; 3, 8 ul 1 mM short peptide RJK001; 4, 8 ul protein solubilization buffer. Use a 20 ul pipette tip to mix them well, then stand still at RT for 20 min.

Quantitative analysis was performed using densitometry of crystallin proteins by western blot analysis of the supernatant or insoluble fraction of lens lysates. Collect one intact eye lenses from cataract patients. Put it in 2 ml Eppendorf tube containing 700 ul protein solubilization buffer, Grind the complete lens at 4° C., 30s once for 3 times with Electric grinder to obtain 1 ml grinding fluid, add 20 ul 2.5 mM RJK001 in 2 ul grinding fluid as the experimental group, add 20 ul protein dissolving solution in 2 ul grinding fluid as a control, place it at RT for 10 min, centrifuge at 13000 rpm and take the supernatant for western blotting, After blocking with 5% milk, membrane were incubated with anti-βB1 crystallin (Santacruz, sc-48335, 1:3000) overnight at 4° C., Membrane were incubated with secondary antibodies for 1 h at room temperature, then the blots were detected by the enhanced chemiluminescence.

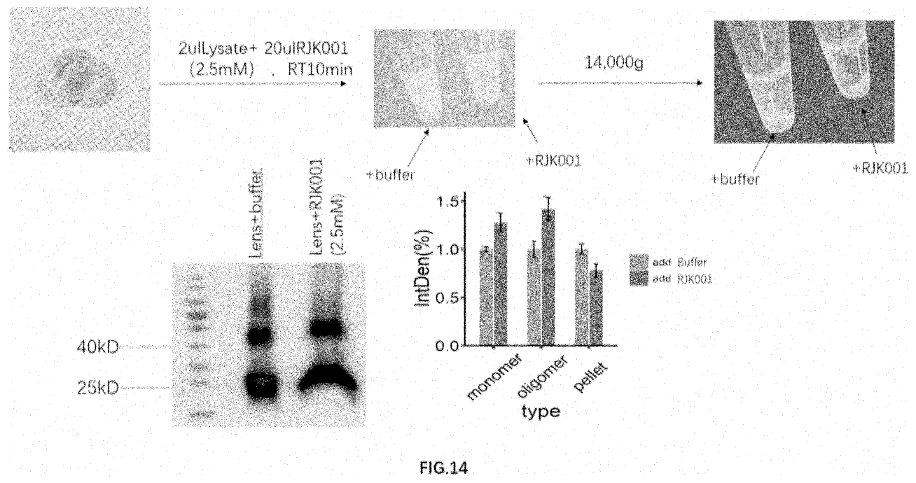

C-terminal fusion cell-penetrating peptide RJK001 and N-terminal fusion cell-penetrating peptide RJK012 were purified in vitro and added into SH-SY5Y cell. Cells were harvested at 1 h, 4 h, 8 h and 24 h respectively and were washed with PBS. Total proteins were extracted from SH-SY5Y cells with lysis buffer. Equal amounts of protein (20 μg) were separated by SDS-PAGE (15% separating gel). For data analysis, ImageJ was used to detect the Integrated density of each protein bands, and the ratio and half-life of Champ peptides were calculated by comparing with the positive control. As shown in FIG. 14, the polypeptides provided herein (e.g., RJK001) could improve the solubility of crystallin proteins of the lens from cataract patients.

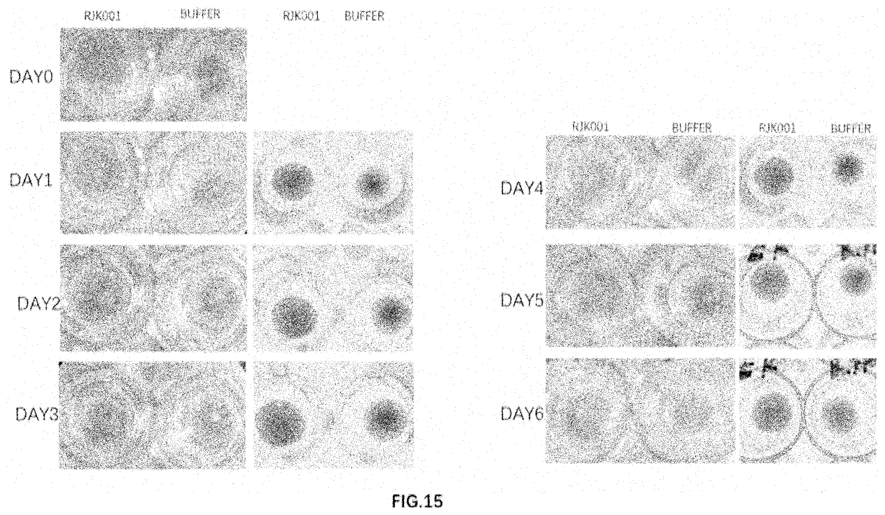

Next, we directly immersed the cataract lens removed during the operation in the buffer solution of the control group and the RJK001 protein solution of the experimental group. During the immersion for up to 6 days, the lens of the patient was photographed and recorded every day. More detailed description of the method is: Collect two intact eye lenses from cataract patients. To each tube was added 400 μL solution of vehicle (0.1% NaN3, 0.3% triton X-100, containing 1:1000 protease inhibitor cocktail) or RJK001 (0.5 mM) in vehicle which covered the lens tissue completely. Lens tissue was incubated in these solutions for 6 days in the dark at room temperature. After sealing the 48-well plate with parafilm to avoid liquid evaporation. Observe the state of the lens under the stereoscope every day and take pictures to record. As shown in FIG. 15, the polypeptides provided herein (e.g., RJK001) reduced opacity of the lens of cataract patients, whereas the buffer could not reduce the opacity of the lens of cataract patients. These data collectively suggested that the polypeptides provided herein are capable of preventing, alleviating or treating protein aggregation (caused by e.g., phase separation) induced visual disorders, such as cataracts.

### Example 5: Methods and Materials Plasmids

Genes of full-length human γD-crystallin (NCBI Reference Sequence: NM_006891.4), the truncations of γD-crystallin(W43R) were inserted into the pCMV7.1 vector with an N-terminal 3×Flag-tag and GFP fused at the C-terminal. The human γD-crystallin gene was cloned into pET14b plasmid. Genes of full-length mouse G3BP1, G3BP1-eGFP, and Champ-E(RJK001) and its variants (Champ-K/Champ-D/Champ-K/Champ-Q) were inserted into the pET-23b vector with a C-terminal His₆-tag and a thrombin protease cleavage site.

### Protein Expression and Purification

All plasmids were expressed in Escherichia coli rosetta (DE3) cells. Cells were grown to an OD₆₀₀ of 0.8 and induced with 0.4 mM IPTG overnight at 16° C. Champ-E and its variants (Champ-K/Champ-D/Champ-K/Champ-Q) were purified with a HisTrap FF column (GE Healthcare) with Tris buffer (50 mM Tris-HCl, 150 mM NaCl, a gradient of ˜0-500 mM imidazole, pH 7.5). The N-terminal Trx1-tag was removed with thrombin protease in the buffer of 50 mM Tris-HCl, 150 mM NaCl, pH 7.5. The cleaved proteins were immediately loaded onto the size-exclusion chromatography column Superdex 75 10/300 (GE Healthcare). The buffer of 50 mM Tris-HCl, 150 mM NaCl, pH 7.5 was used for a Superdex 75 column containing. All proteins were concentrated via centrifugal filtration (Amicon, Millipore) and flash frozen with liquid nitrogen. [00175]γD-crystallin was produced in transformed E. Coli BL21(DE3) cells at 37° C. for 4 hours with 1 mM IPTG. Protein was purified by Ni affinity chromatography In Tris HCl pH 7.5, 150 mM NaCl using a linear gradient of imidazole. All the fractions containing γD-crystallin were pooled and subjected to gel filtration on Supdex75 column (GE Healthcare) for the final purification.

The purified protein was loaded to SDS-PAGE for the purity check.

### In Vitro Phase Separation Assays

### Droplet Assay for Microscopy

For G3BP1(FL)-eGFP, purified G3BP1(FL)-eGFP, Champ-E and its variants were changed were diluted to droplet buffer (50 mM Tris, pH 7.5, 150 mM NaCl) using SpinDesalt Column (smart). G3BP1(FL)-eGFP proteins were diluted to indicated concentrations and supplemented with PEG8000 with 1.25% final concentration for 10 min to induce phase separation as reported [1].

For γD-crystallin, γD-crystallin was purified in vitro, then were dissolved in protein solubilization buffer (150 mM NaCl, 50 mM tris-HCl, PH 7.5) to a final concentration of 50 mg/ml, Place γD-crystallin at 4° C. for 20 minutes to induce phase separation.

### Turbidity Assays

We followed the Turbidity Assays experiment as previously reported [2]. Briefly, G3BP1 turbidity analysis were at room temperature and γD-crystallin protein was at 4° C. Proteins and Champ-E(RJK001), Champ-K, Champ-D or mutants are mixed at room temperature for 10 minutes prior to turbidity assays. 20 μM G3BP1 and 50 mg/ml(2.5 mM) γD-crystallin and peptide were mixed well in a ratio range of 0.75 to 25 before test. Absorbance at 395 nm (OD395 nm) was monitored at room temperature using Nanodrop ONE (Thermo Fisher Scientific, Inc.).

### Nuclear Magnetic Resonance Spectroscopy

NMR were done on a bruker avance III HD spectrometer at 25° C.

For the N15 labeling of the protein, the transformed E. Coli BL21(DE3) cells were cultured and induced in M9 medium with NH₄Cl as the only nitrogen source.

The N15 labeled protein was mix with the unlabeled peptide with the ratio of 1:0, 1:2. HSQC spectrums were acquired and analysis with ccpNMR. Amide ¹H-¹⁵N combined chemical shift differences were calculated using the equation Δδ) [(0.125ΔδN)²+ΔδH²]. Intensity changes were calculated by I/I0. Combined chemical shift perturbations were calculated using the equation based on a previous study.

The amino acids with chemical shift differences higher than 0.1 was highlighted in the structure (2KFB).

### Cell Culture and Transfection

SH-SY5Y cells were plated on 12-well chamber at 1×10⁵ cells per well in medium (DMEM (High Glucose) supplemented with 10% fetal bovine serum and 1% Penicillin/Streptomycin (Invitrogen)). -terminal fusion cell-penetrating peptide RJK001 and N-terminal fusion cell-penetrating peptide RJK012 were purified in vitro and added into SH-SY5Y cell.

For γD (W43R) granule assay, SH-SY5Y cells were cultured in DMEM (High Glucose) supplemented with 10% fetal bovine serum and 1% Penicillin/Streptomycin (Invitrogen). Cells were grown in a 12-well plate (1×10⁵ cells per well) for 24 hours and transfected with γD (W43R) plasmid using EZtrans transfection reagent.

### Quantitative Analyses of Granules in Cells

Granules screen and assay images were segmented and image features quantified using a custom CellProfiler and ImageJ software. Briefly, Live imaging were collected hourly using BioPipeline LIVE real-time observation and analysis system at 37° C. The effect of RJK001 and RJK012 polypeptide on the γD (W43R) aggregates in the SH-SY5Y cell mode were analyzed using a custom CellProfiler software. Briefly, cell bodies were then segmented and identified by the GFP fluorescence channel images and tracing outward to the limits of the cytoplasmic (using a diameter cutoff of 100-300 pixels). The cell bodies were used as masks to eliminate imaging artifacts outside of cell boundaries, such as background fluorescence or dead cells. After masking, punctate structures were enhanced by image processing for speckle-like features that were 10 pixels in diameter for SH-SY5Y cells and these punctate structures were then annotated as features such as γD (W43R) puncta. Finally, the total image area which was enclosed in each of the identified features (number of cell and punctate) was calculated and output to spreadsheets. At least 200 cells per sample were analyzed.

### Western Blotting

Cells were harvested at 1 h, 4 h, 8 h and 24 h respectively and were washed with PBS. Total proteins were extracted from SH-SY5Y cells with lysis buffer (50 mM Tris (pH 7.4), 150 mM NaCl, 1% NP-40, 0.5% sodium deoxycholate, 0.1% SDS and 1 mM PMSF). Equal amounts of protein (20 μg) were separated by SDS-PAGE (15% separating gel). After electrophoresis, the proteins were transferred to nitrocellulose membranes (300 mA for 1.5 hours). The blots were then blocked in 5% nonfat dry milk solution for 1 hour at room temperature. Antibody specific for cell-penetrating peptide was used and incubated overnight at 4° C. For data analysis, quantification of the western blot bands was achieved using the software GELPRO (Media Cybernetics). The ratio and half-life of Champ peptides were calculated by comparing with the positive control. The presented quantitative data were calculated from three independent experiments.

### REFERENCES

1. Guillen-Boixet, J. et al. RNA-Induced Conformational Switching and Clustering of G3BP Drive Stress Granule Assembly by Condensation. Cell 181, 346-361 e317, doi:10.1016/j.cell.2020.03.049 (2020).
2. Yoshizawa, T. et al. Nuclear Import Receptor Inhibits Phase Separation of FUS through Binding to Multiple Sites. Cell 173, 693-705 e622, doi:10.1016/j.cell.2018.03.003 (2018).

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A method for treating phase separation associated disease in a subject in need thereof, comprising administering to the subject in need thereof a therapeutically effective amount of a polypeptide, a polynucleotide encoding the polypeptide, and/or a vector comprising the polynucleotide, the polypeptide comprising a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues, and wherein the polypeptide is capable of reversing phase separation.

2. The method of claim 1, wherein the phase separation associated disease is phase separation associated vision disorder.

3. The method of claim 2, wherein the phase separation associated vision disorder is cataract.

4. The method of claim 1, wherein the hydrophilic segment has a sequence selected from the group consisting of:
(SEQ ID NO: 11)
TX₁PQX₁X₁SX₁X₁X₁VX₁X₁PX₁X₁R,
(SEQ ID NO: 12)
X₁LX₁X₁X₁SX₁X₁X₁VX₁X₁X₁QX₁X₁X₁,
(SEQ ID NO: 13)
X₁X₁X₁VX₁X₁X₁X₁X₁VX₁X₁,
and
(SEQ ID NO: 14)
X₁X₁SX₁VQX₁LX₁,
wherein each X₁ is respectively Asp, Glu, Lys or Arg.

5. The method of claim 1, wherein the hydrophilic segment has a sequence selected from the group consisting of:
(SEQ ID NO: 15)
TEPQEESEEEVEEPEER,
(SEQ ID NO: 16)
TDPQDDSDDDVDDPDDR,
(SEQ ID NO: 17)
TKPQKKSKKKVKKPKKR,
(SEQ ID NO: 18)
TRPQRRSRRRVRRPRRR,
(SEQ ID NO: 19)
ELDEESEDEVEEEQEDR,
(SEQ ID NO: 20)
KEEVDEDRDVDE,
and
(SEQ ID NO: 21)
EKSEQDLE,
or a sequence having at least 90% identity thereto, or a sequence having 1, 2, 3, 4, or 5 amino acid residue difference therefrom.

6. The method of claim 1, wherein the hydrophobic segment has a sequence selected from the group consisting of:
(SEQ ID NO: 22)
TFYDQTVSNDL,
(SEQ ID NO: 23)
ANSAYYDAHPVTNGI,
(SEQ ID NO: 24)
PPQTAAREATSIPGFPAEGAIPLPV,
and
(SEQ ID NO: 25)
EGEVAEEPNSRP,
or a sequence having at least 90% identity thereto, or a sequence having 1, 2, 3, 4, or 5 amino acid residue difference therefrom.

7. The method of claim 1, wherein the polypeptide comprises a sequence selected from the group consisting of:
(SEQ ID NO: 1)
TEPQEESEEEVEEPEERQQTPEVVPDDSGTFYDQTVSNDLE
(RJK001),
(SEQ ID NO: 2)
TDPQDDSDDDVDDPDDRQQTPDVVPDDSGTFYDQTVSNDLD
(RJK002),
(SEQ ID NO: 3)
TKPQKKSKKKVKKPKKRQQTPKVVPDDSGTFYDQTVSNDLK
(RJK012),
(SEQ ID NO: 4)
TRPQRRSRRRVRRPRRRQQTPRVVPDDSGTFYDQTVSNDLR,
(SEQ ID NO: 8)
ELDEESEDEVEEEQEDRQPSPEPVQENANSAYYDAHPVINGIE,
(SEQ ID NO: 9)
KEEVDEDRDVDESSPQDSPPSKASPAQDGRPPQTAAREATSIPGFPAEG
AIPLPV,
and
(SEQ ID NO: 10)
EGEVAEEPNSRPQEKSEQDLE,
or a sequence having at least 90% identity thereto, or a sequence having 1, 2, 3, 4, or 5 amino acid residue difference therefrom.

8. The method of claim 1, wherein the hydrophilic segment has a length of 10-17 amino acid residues among which at least 60% are Asp, Glu, Lys, or Arg,
wherein the hydrophobic segment having a length of 10-12 amino acid residues among which at least 35% are Tyr, Phe, Leu, or Val, and
wherein the polypeptide has a length of 20-28 amino acid residues.

9. The method of claim 8, wherein the hydrophilic segment has a sequence of:
(SEQ ID NO: 11)
TX₁PQX₁X₁SX₁X₁X₁VX₁X₁PX₁X₁R,
wherein each X₁ is Asp, Glu or Lys.

10. The method of claim 8, wherein the hydrophobic segment has a sequence of:
TFYDQTVSNDL (SEQ ID NO: 22),
or a sequence having at least 90% identity thereto, or a sequence having 1, 2, 3, 4, or 5 amino acid residue difference therefrom.

11. The method of claim 8, wherein the polypeptide comprises a sequence of:
(SEQ ID NO: 31)
TX₁PQX₁X₁SX₁X₁X₁VX₁X₁PX₁X₁RQQTPX₁VVPDDSGTFYDQTVSNDLX₁,
wherein each X₁ is Asp, Glu or Lys.

12. The method of claim 1, wherein the polypeptide is fused with a cell-penetrating peptide.

13-14. (canceled)

15. The method of claim 1, wherein the polypeptide is further fused with a linker.

16. The method of claim 15, wherein the linker comprises a sequence selected from the group consisting of:
(SEQ ID NO: 28)
SGRPVL,
(SEQ ID NO: 29)
GAPGSAGSAAGGSG,
and
(SEQ ID NO: 30)
ENLVFQG.

17. The method of claim 1, wherein the polypeptide is further fused with a his tag.

18-19. (canceled)

20. The method of claim 1, wherein the polypeptide, the polynucleotide encoding the polypeptide, and/or a vector comprising the polynucleotide is administered orally, intravenously, intramuscularly, enterally, mtraocularly, subretinally, intravitreally, topically, ocularly (eye drops, insert, injection or implant), sublingually, rectally or by injection, nasal spray or inhalation.

21. (canceled)

22. The method of claim 1, wherein the polypeptide, the polynucleotide encoding the polypeptide, and/or a vector comprising the polynucleotide is formulated as an ophthalmic solution, an ophthalmic ointment, an ophthalmic wash, an intraocular infusion solution, a wash for anterior chamber, an internal medicine, an injection, an intravitreal injection, an anterior chamber injection, a subarachnoid injection or preservative for extracted cornea.

23. A method for:
(a) dissolving a crystalline protein aggregate and/or preventing the formation of crystalline protein aggregate in a cell, comprising introducing to the cell a polypeptide; and/or
(b) inhibiting, alleviating, and/or preventing phase separation of crystallin protein, comprising contacting the crystallin protein with a polypeptide;
wherein the polypeptide comprises a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues, and wherein the polypeptide is capable of inhibiting phase separation.

24. The method of claim 23, wherein the crystalline protein aggregate is βD-crystallin aggregate, γD-crystallin aggregate, or a combination thereof.

25. A kit for treating and phase separation associated diseases (e.g., cataracts), comprising a formulation of a therapeutically effective amount of a polypeptide, a polynucleotide encoding the polypeptide, and/or a vector comprising the polynucleotide, a pharmaceutically acceptable carrier and instructions for administering the formulation such that the administration treats the phase separation associated diseases,
wherein the polypeptide comprises a hydrophilic segment and a hydrophobic segment,
wherein the hydrophilic segment has a length of 10-20 amino acid residues among which at least 50% are Asp, Glu, Lys, or Arg,
wherein the hydrophobic segment having a length of 10-20 amino acid residues among which at least 50% are Tyr, Phe, Trp, Leu, Ile, Val, Met, Pro, Ala, or Cys,
wherein the hydrophilic segment is at the N-terminus and the hydrophobic segment is at the C-terminus, or vice versa,
wherein the polypeptide has a length of 20-60 amino acid residues, and wherein the polypeptide is capable of inhibiting phase separation.

26-27. (canceled)
